(19) United States
(12) Patent Application Publication
(10) Pub. No.: US 2002/0025296 A1
(43) Pub. Date: Feb. 28, 2002

(54) Combined use of nucleoside analogues and gene transfection for tissue imaging and therapy

(76) Inventors: Edward E. Knaus, Edmonton (CA); Leonard I. Wiebe, Edmonton (CA); Kevin Morin, Edmonton (CA)

Correspondence Address: RODMAN RODMAN, 7 SOUTH BROADWAY, WHITE PLAINS, NY, 10601

(21) Appl. No.: 09/855,176

(22) Filed: May 14, 2001

Related U.S. Application Data
(63) Continuation-in-part of application No. 08/836,586, filed on Jul. 14, 1997, now abandoned.

(30) Foreign Application Priority Data
Oct. 21, 1994 (GB) 9421223.0
Oct. 20, 1995 (CA) PCT/CA95/00593

(51) Int. Cl. A61K 51/06; C12Q 1/68; A61K 48/00

(52) U.S. Cl. 424/1.17; 435/6; 514/44

(57) ABSTRACT

A method and use of a labelled compound for monitoring the transfer of a foreign gene including selecting the foreign gene which has been isolated from a cell or virus and transferred into a cell population and selecting the labelled compound which will interact selectively with a protein expressed by the foreign gene to produce a labelled product. The labelled compound has a rate of expulsion from the cells which is greater than that of the labelled product. Further, the use and method include administering to the cells an effective dose of the labelled compound such that the labelled compound selectively interacts with the protein to produce the labelled product, waiting a period of time such that a substantial amount of the labelled compound has been expelled from the cells and such that a detectable amount of the labelled product remains and determining the extent and location of the protein by detecting the labelled product.

### TECHNICAL FIELD

This invention relates to diagnostic, radiotherapy and chemotherapy methods for use in conjunction with gene therapy techniques and to the use of certain compounds in performing these methods.

### BACKGROUND ART

The utilization of gene therapy techniques to express foreign proteins within tissues and cell populations is providing insights into their function and plasticity. These techniques have been successfully used to investigate and treat a broad range of physiological processes. Progress in manipulating transgenic products in vivo and achieving cell-specific delivery of genetic material provides encouragement for enhancing the value of these techniques and their therapeutic potential for treating human and animal disorders.

One aspect of gene therapy involves the transfer of DNA to introduce a sensitivity gene into a target tissue. This can be achieved by direct injection of the DNA into the target tissue, delivery of DNA via liposomes, or via a viral vector that transfers the gene to the target tissue. In the latter case, the viral vector is genetically modified to include the new sensitivity gene in its genome. Such vectors are capable of “transducing” mammalian cells, resulting in expression of a protein which is encoded by the new gene. This expressed protein sensitizes the target tissue to a drug which is a substrate for the protein expressed. The enzymatic process induced by the drug leads to death of target tissue cells expressing the protein. Since proteins that are present in non-transduced cells have a very low affinity for the drug, systemic toxicity related to this mechanism is not observed.

Gene transfer can be retrovirus-mediated, which provides gene integration only into target cells that are actively synthesizing DNA, with the result that adjacent non-proliferating normal target tissue or cells should not acquire the gene and should remain insensitive to the drug. All of the transduced target cells and viral vector producing cells will be killed by the drug treatment and/or host immune response eliminating potential concern regarding insertional mutagenesis that could give rise to malignant cells.

Gene therapy has been used to treat malignant tumors by in vivo genetic manipulation of the tumor's genome. For example, the efficacy of the herpes simplex virus type 1 thymidine kinase (HSV-1 TK) gene as a “suicide vector” in gene therapy of cancer has been demonstrated [M. P. Short et al., J. Neurosci. Res., 27, 427 (1990); Y. Takamiya et al., J. Neurosurg. 79, 104 (1993)]. One of the most promising approaches involves HSV-1 TK gene transduction in brain tumors followed by intravenous ganciclovir {9-[1,3-dihydroxy-2-propoxy)methyl]guanine, GCV} treatment which is selectively toxic to transduced cells due to selective phosphorylation by HSV-1 TK [K. W. Culver et al., Science, 256, 1550 (1992)]. GCV-monophosphate is subsequently converted by endogenous mammalian kinases to GCV-triphosphate which is a potent inhibitor of viral DNA polymerase. In this therapy, delivery of the Moloney murine retrovirus vector has been achieved through stereotactic implantation of mouse fibroblast producer cells into the brain tumor mass. Transduction efficiency and subsequent expression of HSV-1 TK in neoplastic tissue is variable and optimal treatment time is currently unknown. However, the dramatic tumor regressions (or cures) observed in animal models, and the lack of systemic toxicity, has prompted the initiation of clinical trials in humans [E. H. Oldfield et al., Hum. Gene Ther., 4, 60 (1993); S. M. Freeman et al., Hum. Gene Ther., 3, 342 (1992)].

Other studies have also indicated the value of gene therapy techniques in the treatment of cancers [F. L. Moolten et al., J. Natl. Cancer Inst, 82, 297 (1990); S. Freeman et al., Cancer Res., 53, 5274 (1993); D. Barba et al., Proc. Nad. Acad. Sci, U.S.A., 91, 4348 (1994)], including types of cancer such as human lung cancers [Y. Hasegawa et al., Am. J. Resp. Cell and Mol Biol. 8, 655 (1993)] and breast cancers [Y. Manome et al., Cancer Res., 54, 5408 (1994)]. Related studies using adenovirus HSV-TK gene transfer into cancer cells has also provided encouraging results for thoracic (W. Roy Symthe et al., Cancer Res., 54, 2055 (1994)], brain [S. -H. Chen et al., Proc. Natl. Acad. Sci. U.S.A., 91, 3054 (1994)] and head and neck [B. W. O'Malley et al., Cancer Res., 56, 1080 (1995)] cancers. It has also been suggested that an adenovirus vector encoding for HSV-TK in porcine arteries produces cells that are sensitive to GCV treatment and may have value to limit smooth muscle cell proliferation in response to arterial injury [T. Ohno et al., Science, 265, 781 (1995)]. However, it has also been shown that 5-(thien-2-yl)- and 5-(furan-2-yl)-2′-deoxyuridine are at least 100-fold more cytostatic to HSV-TK gene-transfected FM3A cells than wild-type FM3A/O cells, and that viral TK expressed in the HSV-1 TK gene-transfected tumor cells merely acts as an activating enzyme, whereas thymidylate synthase serves as the target enzyme for the cytostatic action of these compounds [C. Bohman et al., J. Biol Chem., 269, 8036 (1994)]. Other viral genes may be employed similarly for gene therapy, in conjunction with an anti-viral nucleoside in a prodrug form that is biotransformed to a cytotoxic form by the protein encoded by the selected transfected viral gene.

One significant limitation associated with any gene therapy technique is that one cannot be certain that gene transfer has been restricted to the tumor or other target tissue, and that it has not also occurred in other sensitive dividing cells such as those of bone marrow or intestinal lining. A second major limitation is that even in the target tissue, gene transfer does not necessarily mean that the gene is actually expressed to give the active protein throughout the target tissue. Currently, an invasive technique, which requires obtaining a biopsy sample of the transduced target tissue, is used to determine the extent of gene transfer by employing an in vitro technique such as beta-galactosidase staining [Z. Ram et al., Cancer Res., 53, 83 (1993); R. G. Vile et al., Cancer Res., 53, 962 (1993)]. Other techniques such as Doppler color-flow and ultrasound imaging only provide images of the tumor vasculature and tumor volume but no information regarding gene transfer efficacy [Z. Ram et al., J. Neurosurg., 81, 256 (1994)]. Morphological imaging techniques such as computed tomography (CT) and magnetic resonance imaging (MRI) also do not provide information regarding gene transfer efficacy.

Accordingly, there is a need for a diagnostic method which may be used in conjunction with gene therapy techniques to monitor the transfer of a foreign gene throughout a population of cells including target tissue, which method when performed in vivo is preferably non-invasive. More particularly, there is a need for a method for monitoring the transfer of foreign genes which are actively expressing a protein. There is also a need to identify compounds possessing specific properties which are suitable for use in performing this diagnostic method. Such a diagnostic method and the use of such compounds in performing this diagnostic method may also be adapted to satisfy a need for radiotherapy and chemotherapy methods which may be used in like manner in conjunction with gene therapy techniques.

### DISCLOSURE OF INVENTION

The present invention provides for a diagnostic method, a radiotherapy method, and a chemotherapy method, which methods may be used in conjunction with gene therapy techniques. The invention also provides for the use of labelled and unlabelled compounds in performing these methods, which labelled and unlabelled compounds have specific physical and chemical properties.

The invention is applicable to populations of cells into which a foreign gene has been transferred, which foreign gene expresses a protein which preferably is not naturally occurring within the cells. A compound is chosen which will interact selectively with the protein expressed by the foreign gene to produce a product which is trapped within the cells, is cytotoxic or cytostatic to the cells, or both, depending upon whether the compound is being used for diagnostic purposes or for radiotherapy or chemotherapy purposes.

In the case of diagnostic applications, trapping of the product, which is labelled, permits the product to accumulate in those of the cells in which the protein has been expressed by the foreign gene, thus facilitating detection of the labelled product in those cells. Thus, the labelled compound is selected to interact selectively with the protein expressed by the foreign gene to produce the labelled product and is further selected to have a rate of expulsion or clearance from the cells which is greater than a rate of expulsion or clearance from the cells of the labelled product.

In the case of radiotherapy applications, trapping of the product, which is radioactive as a result of the compound being radiolabelled, permits the product to accumulate in those of the cells in which the protein has been expressed by the foreign gene, thus facilitating radiotherapeutic effects directed specifically at those cells. In the case of chemotherapy applications, interaction of the protein with the compound has a cytotoxic or cytostatic effect on the cells, which is enhanced if the product is trapped within those of the cells in which the protein has been expressed.

In a first embodiment relating to diagnostic applications of the invention, the invention comprises a method for monitoring the transfer of a foreign gene throughout a population of cells, comprising the steps of administering to the cells an effective dose of a labelled compound so that the labelled compound interacts selectively with a protein expressed by the foreign gene to produce a labelled product, and then detecting the labelled product, wherein the labelled compound is selected to interact selectively with the protein expressed by the foreign gene such that the labelled product becomes trapped within those of the cells in which the protein has been expressed by the foreign gene. In this first embodiment, the invention also comprises the use of labelled compounds in performing this diagnostic method.

In a preferred diagnostic method, the method for monitoring the transfer of the foreign gene throughout the population of cells is comprised of the following steps:

(a) selecting a foreign gene which has been isolated from a cell or virus and which has been transferred into the population of cells;

(b) selecting a labelled compound which will interact selectively with a protein expressed by the foreign gene to produce a labelled product and which has a rate of expulsion from the cells which is greater than a rate of expulsion from the cells of the labelled product;

(c) administering to the cells an effective dose of the labelled compound such that the labelled compound selectively interacts with the protein to produce the labelled product;

(d) waiting a period of time such that a substantial amount of the labelled compound has been expelled from the cells and such that a detectable amount of the labelled product remains within the cells; and

(e) determining the extent and location of the protein throughout the population of cells by detecting the labelled product.

The method may further comprise the steps of isolating the selected foreign gene from a cell or virus and transferring the isolated foreign gene into the population of cells. In addition, the invention also comprises the use of labelled compounds in performing this preferred diagnostic method.

The foreign gene may be a gene selected from any eucaryotic or procaryotic cells, or from a virus, including a virus from the group of viruses consisting of herpes simplex virus, human cytomegalovirus, varicella zoster virus and Epstein-Barr virus. A preferred foreign gene is a gene which expresses herpes simplex virus thymidine kinase.

The labelled compound is preferably radiolabelled, but any other form of labelling which facilitates detection of the labelled product may also be suitable. Where the foreign gene is a gene which expresses herpes simplex virus thymidine kinase, the preferred labelled compound is a compound of the following formula:

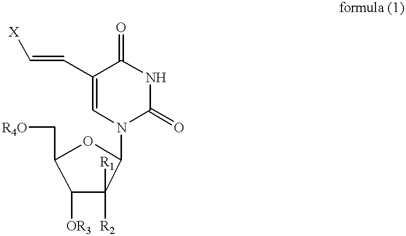

or a pharmaceutically acceptable salt thereof, wherein X is a radioactive halogeno substituent, wherein R₁ is a hydrogen, hydroxyl or fluoro substituent, wherein R₂ is a hydrogen or fluoro substituent, wherein R₃ is a substituent selected from the group consisting of hydrogen, arylcarbonyl, heteroarylcarbonyl, heterocyclocarbonyl, 1-methyl-1,4-dihydropyridyl-3-carbonyl, 3-7C cycloalkylcarbonyl, and alkylcarbonyls with a straight or branched chain having from 1 to 8 carbon atoms, and wherein R₄ is a substituent selected from the group consisting of hydrogen, arylcarbonyl, heteroarylcarbonyl, heterocyclocarbonyl, 1-methyl-1,4-dihydropyridyl-3-carbonyl, 3-7C cycloalkylcarbonyl, and alkylcarbonyls with a straight or branched chain having from 1 to 8 carbon atoms.

Preferably, at least one of R₃ and R₄ are hydrogen, and most preferably, R₄ is hydrogen. Preferably X is selected from the group consisting of ¹²³I, ¹²⁴I, ¹³¹I, ⁷⁵Br, and ¹⁸F, and most preferably X is ¹²³I. The substituents for some specific preferred labelled compounds for use with this foreign gene are as follows:

1. X=¹²³I ¹²⁴I or ¹³¹I, most preferably ¹²³I

R₁=hydrogen

R₂=hydrogen

R₃=hydrogen or 1-methyl-1,4-dihydropyridyl-3-carbonyl

R₄=hydrogen

2. X=¹²³I, ¹²⁴I or ¹³¹I, most preferably ¹²³I

R₁=hydrogen

R₂=fluorine

R₃=hydrogen or 1-methyl-1,4-dihydropyridyl-3-carbonyl

R₄=hydrogen

3. X=¹²³I, ¹²⁴I or ¹³¹I, most preferably ¹²³I

R₁=fluorine

R₂=hydrogen

R₃=hydrogen or 1-methyl-1,4-dihydropyridyl-3-carbonyl

R₄=hydrogen

4. X=¹²³I, ¹²⁴I or ¹³¹I most preferably ¹²³I

R₁=hydroxyl

R₂=hydrogen

R₃=hydrogen or 1-methyl-1,4-dihydropyridyl-3-carbonyl

R₄=hydrogen

In the case of diagnostic applications, metabolic trapping of the labelled product as compared to the labelled compound permits the labelled product to accumulate in those of the cells in which the protein has been expressed by the foreign gene while the labelled compound is expelled, excreted or otherwise cleared from the cells by metabolic or other biological processes in order to facilitate the detection of the labelled product in those cells. Thus, the labelled compound is particularly selected to interact selectively with the protein expressed by the foreign gene to produce the labelled product and is further selected to have a rate of expulsion or clearance from the cells which is greater than a rate of expulsion or clearance from the cells of the labelled product.

Accordingly, following the administration of an effective dose of the labelled compound to the cells such that the labelled product is produced in the cells as a result of the selective interaction of the labelled compound with the protein expressed by the foreign gene, the diagnostic method includes waiting a period of time such that a substantial amount of the labelled compound has been expelled from the cells and such that a detectable amount of the labelled product remains within the cells.

The period of time for waiting, prior to performing the step of determining the extent and location of the protein throughout the cells by detecting the labelled product, will be determined or selected depending upon a number of various factors including the properties of each of the labelled compound and the labelled product, as well as the selected method or process for detecting the labelled product. For instance, the rate of expulsion or clearance of each of the labelled compound and the labelled product from the cells will be a primary determinative factor. The time period is selected to achieve a balance between the amount of the labelled compound present in the cells and the amount of the labelled product present in the cells at the time of detecting the labelled product. First, the amount of the labelled compound is preferably minimized in order to enhance or increase the accuracy of the diagnostic method as the presence of significant or substantial amounts of the labelled compound may interfere with the detection of the labelled product. For instance, in radiolabelling of the compound and the product, radioimaging may be unable to distinguish between the presence of the labelled compound in the cells as compared with the labelled product. Second, the amount of the labelled product within the cells is preferably maximized to facilitate the detection of the labelled product and to also enhance or increase the accuracy of the diagnostic method.

Most preferably, the labelled compound and the labelled product have relative rates of clearance such that following the passage of a determined or selected period of time, all or substantially all of the labelled compound has been expelled from the cells while all or substantially all of the labelled product remains within the cells. In other words, the expulsion of the labelled compound and the expulsion of the labelled product do not overlap such that the expulsion of the labelled compound from the cells is complete or substantially complete prior to the commencement of any expulsion or any substantial expulsion of the labelled product from the cells.

However, the relative rates of clearance may provide for an overlap of the expulsion of the labelled compound and the expulsion of the labelled product from the cells. In this case, the period of time is selected or determined according to the desired degree of accuracy or the desired statistical significance of the diagnostic test results as discussed above. As indicated, the period of time is selected so that preferably a substantial amount of the labelled compound has been expelled. For a substantial amount to be expelled, any remaining labelled compound in the cells is not enough to significantly interfere with the detection of the labelled product and is such that the diagnostic test results achieve the desired degree of accuracy or statistical significance. The period of time is also selected so that a detectable amount of the labelled product remains within the cells. A detectable amount is present if there is a sufficient amount to permit effective detection according to the selected detection method or process and such that the diagnostic test results achieve the desired degree of accuracy or statistical significance. For instance, where radiolabelling and radioimaging are used, a sufficient amount of the labelled product must remain in the cells to provide adequate signal measurement.

Once this period of time has passed, the extent and location of the protein expressed by the foreign gene throughout the population of cells is determined by detecting the labelled product. The determination of the extent and location of the protein in the cells provides for or permits the monitoring of the transfer of foreign genes which are actively expressing the protein into the cells in which those foreign genes are located. The location of the protein refers to the place or site where the protein is found or is present within the cell population. The extent of the protein refers to a quantitative measurement of the degree to which the protein extends throughout or within the cell population or the length, area, volume or scope of the protein throughout the cell population.

The method of detection is selected according to the type or manner of the labelling of the product. However, in the preferred embodiment, the labelled product is radiolabelled and the detection is performed using nuclear medicine imaging techniques.

In a second embodiment relating to radiotherapy applications of the invention, the invention comprises a method of radiotherapy for use with a population of cells into which a foreign gene has been transferred, comprising the step of administering to the cells an effective radiotherapeutic dose of a radiolabelled compound so that the radiolabelled compound interacts selectively with a protein expressed by the foreign gene to produce a radiolabelled product, wherein the radiolabelled compound is selected to interact selectively with the protein expressed by the foreign gene such that the radiolabelled product becomes trapped within those of the cells in which the protein has been expressed by the foreign gene. In this second embodiment, the invention also comprises the use of radiolabelled compounds in performing this radiotherapy method.

The parameters for choosing a foreign gene for use in the radiotherapy method of the invention are the same as for the diagnostic method of the invention, and a preferred foreign gene is a gene which expresses herpes simplex virus thymidine kinase.

Where the foreign gene is a gene which expresses herpes simplex virus thymidine kinase, the preferred radiolabelled compound is of the same general formula (1) as for the diagnostic method of the invention, except that X is a radioactive halogeno substituent, preferably selected from the group consisting of ¹²³I, ¹²⁵I and ¹³¹I. Most preferably X is ¹³¹I. Preferably, at least one of R₃ and R₄ are hydrogen and most preferably R₄ is hydrogen. The substituents for the specific preferred radiolabelled compounds for use with this foreign gene are the same as for the diagnostic method of the invention, except that X in these specific radiolabelled compounds is selected from the group consisting of ¹²³I, ¹²⁵I and ¹³¹I.

In a third embodiment relating to chemotherapy applications of the invention, the invention comprises a method of chemotherapy for use with a population of cells into which a foreign gene has been transferred, comprising the step of administering to the cells an effective chemotherapeutic dose of a compound so that the compound interacts selectively with a protein expressed by the foreign gene to produce a product, wherein the compound is selected to interact selectively with the protein expressed by the foreign gene such that the product is cytotoxic or cytostatic to those of the cells in which the protein has been expressed by the foreign gene. In this third embodiment, the invention also comprises the use of compounds in performing this chemotherapy method.

The parameters for choosing a foreign gene for use in the chemotherapy method of the invention are the same as for the diagnostic method of the invention, and a preferred foreign gene is a gene which expresses herpes simplex virus thymidine kinase.

Where the foreign gene is a gene which expresses herpes simplex virus thymidine kinase, the preferred compound is of the same general formula (1) as for the diagnostic method of the invention, except that X is a halogeno substituent, preferably selected from the group consisting of bromo, chloro, fluoro and iodo. Most preferably X is either a bromo, chloro, or iodo substituent. Preferably, at least one of R₃ and R₄ are hydrogen, and most preferably R₄ is hydrogen. The substituents for the specific preferred compounds for use with this foreign gene are the same as for the diagnostic method of the invention, except that in these specific preferred compounds, X may be described generally as an iodo substituent.

### BRIEF DESCRIPTION OF DRAWINGS

Embodiments of the invention will now be described with reference to the accompanying drawings, in which:

FIG. 1 is a graph showing the in vitro cellular uptake of [¹²⁵I]-IVDU in KBALB-LNL and KBALB-STK cells for example 12;

FIG. 2 is a graph showing the in vitro cellular uptake of [¹²⁵I]-IVFRU in KBALB, KBALB-LNL, and KBALB-STK cells for example 12;

FIG. 3 is a graph showing the in vivo biodistribution of [¹³¹I]-IVFRU in mice bearing KBALB-STK tumors for example 13;

FIG. 4 is a graph showing the in vivo biodistribution of [¹³¹I]-IVFRU in mice bearing KBALB tumors for example 13;

FIG. 5 is a graph showing the in vivo tumor to blood ratios in mice bearing KBALB or KBALB-STK tumors for example 13;

FIG. 6 is a scintigram image of KBALB-STK tumor expressing HSV-1 TK at 8 hours after injection of [¹³¹I]-IVFRU, but before Ganciclovir treatment, for example 14; and

FIG. 7 is a scintigram image of KBALB-STK tumor expressing HSV-1 TK at 8 hours after injection of [¹³¹I]-IVFRU following 4 days of Ganciclovir treatment for example 14.

### BEST MODE FOR CARRYING OUT THE INVENTION

In a first embodiment of the invention, the invention is comprised of a diagnostic use of a labelled compound and a diagnostic method using that compound. The use and the method are for monitoring the transfer of a foreign gene throughout a population of cells. The method is comprised of the steps of first administering an effective dose of a labelled compound to the cells in order to produce a labelled product by the selective interaction described below and then detecting the labelled product.

The foreign gene is preferably deliberately transferred, transduced or transfected into the population of cells or a portion of the population of cells, as desired, by gene therapy techniques. The foreign gene is encoded to express a protein. When an effective dose of the labelled compound is administered to the cells including the transferred foreign gene, the labelled compound interacts selectively with the expressed protein to produce the labelled product. Transfer of the foreign gene into the cells does not necessarily mean that every transferred foreign gene is actively expressing the protein into the cell in which that specific foreign gene is located. Some foreign genes may be actively expressing the protein, while others may be dormant. As a result, the labelled product is produced only within those cells in which the protein has actually been expressed by the foreign gene. The labelled product may then be detected in order to monitor the transfer of the foreign gene in the cells.

The labelled compound is selected to interact selectively with, or be acted on by, the specific protein expressed by the foreign gene in order to produce a labelled product which is trapped and thus localized within, and which does not readily escape from, the cells in which the protein has been expressed. Thus a preferential accumulation or localization or a selective metabolic trapping of the labelled product occurs in the protein expressing cells, as compared to cells which either do not include the foreign gene or which include a dormant foreign gene. This selective trapping permits the specific detection of those cells which both include the foreign gene and in which the specific protein has been expressed. Modification of the labelled compound, such as by phosphorylation, occurs in the presence of the protein, which results in the formation of the labelled product inside the cell. The resulting labelled product does not readily leave the cell and therefore accumulates or is localized within that cell. The labelled product that is trapped within the cell may be any product resulting from the interaction which satisfies the above noted requirements, and which includes the label, as well as the label itself in isolation when the label alone is capable of being selectively trapped in the cell.

As indicated, the process of metabolic trapping of the labelled product provides for the accumulation and retention of the labelled product in those of the cells in which the protein has been expressed by the foreign gene, while the labelled compound is more readily expelled from the cells. To achieve the desired metabolic trapping, the labelled compound is particularly selected to selectively interact with the protein to produce the labelled product and is further selected to have a rate of expulsion or clearance from the cells which is greater than a rate of expulsion or clearance from the cells of the labelled product.

In performing the diagnostic method, the metabolic trapping of the labelled product and the differing rates of expulsion or clearance of the labelled compound and the labelled product are utilized by waiting a period of time prior to determining the extent and location of the protein by detecting the labelled product. Waiting a predetermined period of time permits the formation and accumulation of the labelled product within the cells and the expulsion of a substantial amount of the labelled compound from the cells. The period of time is also determined or selected such that a detectable amount of the labelled product remains trapped within the cells at the time of detecting the labelled product. More particularly, the time period is determined or selected to achieve a balance between the amount of the labelled compound present in the cells and the amount of the labelled product present in the cells at the time of detecting the labelled compound. A time period is preferably selected which maximizes the amount of the labelled compound expelled from the cells while minimizing the amount of the labelled product expelled from the cells. Further, the time period is selected to achieve a desired degree of accuracy or a desired statistical significance of the diagnostic test results. Thus, any labelled compound which has not been expelled from the cells following the waiting period is preferably not significant enough to interfere with or negatively affect the test results. Further, any labelled product remaining within the cells following the waiting period is preferably sufficient to provide the desired test results.

The diagnostic use and method may be used both in vitro and in vivo to monitor the transfer of the foreign gene throughout the population of cells. More specifically, the method may be useful to determine the location or site, the extent and the kinetics of the transfer of the foreign gene throughout the cells, to determine the optimal time for initiation of chemotherapy in mammalian subjects using a prodrug to destroy gene transduced tumors in cancer treatment, to effect radiotherapy of a specific population of cells using radiopharmaceuticals, or to study gene modulation processes. Further, the diagnostic method may be used in clinical studies to assess treatment efficacy and whether repeat treatment is required in the event that regrowth occurs in the cellular population.

Accordingly, the population of cells of the within invention may be any in vitro or in vivo population of cells or a smaller specific portion of a larger cellular population. In the preferred embodiment, the population of cells is either a specific tissue of a mammalian subject, such as a human (eg: brain, liver, breast, etc.) or a portion of a specific tissue, such as a cancerous tumor within the tissue. Given the manner of the foreign gene transference or integration into the cells, the diagnostic method is particularly useful when the cells or tissue in which the transference of the foreign gene is to be monitored are actively synthesizing DNA, as is found in human conditions resulting in cell proliferation. Such cell proliferative conditions include cancer and the repair of tissues after injuries, such as smooth muscle cell proliferation in response to arterial injury.

The transferred gene may be any gene selected from any eucaryotic or procaryotic cell or any virus, which is preferably readily uptaken by the population of cells in which the transference of the foreign gene is to be monitored. Further, the gene must express a protein which, preferably, is not naturally occurring within that population of cells. For this reason, the transferred gene is preferably a foreign gene. A foreign gene is any gene which is not present in that exact or specific form in the population of cells in which the transference of the foreign gene is to be monitored. In other words, a foreign gene is either not present at all in those cells or is present in the cells in a differing form such that the protein expressed by the foreign gene is not naturally expressed in those cells. The protein expressed by the foreign gene may be an enzyme.

As stated, the foreign gene is preferably readily uptaken by the cells into which the foreign gene is to be transferred. For this reason, the foreign gene is preferably selected from a virus. Further, the foreign gene is preferably selected from the group of viruses consisting of herpes simplex virus, human cytomegalovirus, varicella zoster virus and Epstein-Barr virus. Of these viruses, the herpes simplex virus is presently commonly used for the selection of a gene for use in gene therapy. Therefore, in the preferred embodiment of the within diagnostic method and use, the foreign gene is selected from the herpes simplex virus, and more specifically, the herpes simplex virus type 1. The specific foreign gene of the preferred embodiment expresses herpes simplex virus thymidine kinase (HSV-TK). However, as indicated previously, any foreign gene may be used as long as the protein expressed by the foreign gene is matched with an appropriate labelled compound to selectively interact with to produce the required labelled product.

The compound may be labelled by any suitable conventional means as long as the interaction with the expressed protein results in a labelled product which is capable of detection in the cells, preferably in a non-invasive manner. In the preferred embodiment, the labelled compound is radiolabelled and upon the selective interaction of the radiolabelled compound with the expressed protein, a radiolabelled product is produced. The radiolabelled product is detected in the preferred embodiment using known nuclear medicine imaging technology. The specific radioactive label used to radiolabel the compound is selected with reference to the specific type of imaging technology to be used for detection of the resulting radiolabelled product, which selection process is known to those skilled in the art. For example, for single photon emission computed tomography (SPECT), the radioactive isotopes ¹²³I and ¹³¹I are both suitable as labels, although, for clinical use, ¹²³I, is preferred. For positron emission tomography (PET), suitable labels include the radioactive isotopes 124I, ⁷⁵Br, ⁷⁶Br and ¹⁸F.

For many foreign genes, any suitably labelled nucleoside or nucleobase may be chosen as the compound. For other foreign genes, a compound other than a nucleoside or nucleobase may be appropriate. As discussed above, to be considered to be suitable, the protein expressed by the transferred foreign gene and the labelled nucleoside or nucleobase must selectively interact to produce a labelled product which is trapped within those of the cells in which the protein has been expressed. For example, the nucleoside 5-fluoro-2′-deoxyuridine, suitably labelled, may be employed for detecting cells including a gene expressing thymidylate synthase, or fluorocytosine may be employed for cells including a gene expressing cytosine deaminase.

As well, different compounds may possess different biodistribution properties and may therefore have different efficacies or other characteristics which make a particular compound more suitable for detecting and imaging a specific population of cells (e.g., brain, lung, breast, ovary, colon, pancreas, etc.). Therefore, when selecting the compound, consideration should also be given to the properties of that particular compound as they relate to the specific cellular population in which the transfer of the foreign gene is to be monitored.

In the preferred embodiment, in which the transferred foreign gene expresses HSV-TK, a compound of the following formula (1), or a pharmaceutically acceptable salt thereof, is preferably used:

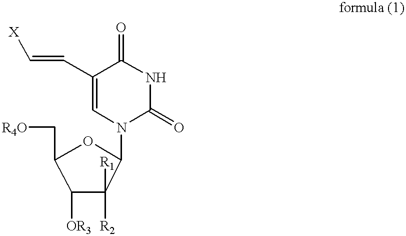

Referring to formula (1), X is the label of the compound, which is preferably a radiolabel as previously described. The radiolabel is preferably a radioactive halogeno substituent. The radioactive halogeno substituent is selected, as stated above, with reference to the type of nuclear medicine imaging technology to be used for detection of the radiolabelled product. Suitable radioactive halogeno substituents include ¹²³I, ¹²⁴I, ¹²⁵I, ¹³¹I, ⁷⁵Br, ⁷⁶Br and ¹⁸F. Of this group, ¹²⁵I is least preferred for diagnostic purposes, ¹²⁴I and ¹³¹I are more preferred, while 1231 is most preferred for clinical use. Therefore in the preferred embodiment, X in the compound of formula (1) is ¹²³I. The radiolabels are incorporated into the compound by conventional techniques.

When X is a radioactive halogeno substituent, the compound of formula (1) is preferably a high-specific-activity no-carrier-added compound. In other words, in the preferred embodiment, the concentration of the radioactive ¹²³I is as high as possible relative to any source iodine or iodine salt contained in the compound in order to increase the specific activity.

R₁ of formula (1) is a hydrogen, hydroxyl or fluoro substituent and R₂ is a hydrogen or fluoro substituent. It has been found that compounds of formula (1) that possess a R₁ or R₂ substituent other than hydrogen at the C-2′ position of the sugar moiety are more stable to pyrimidine phosphorylase which cleaves the glycosidic C—N bond of most C-2′ unsubstituted pyrimidine nucleosides. For example, it is known that a 2′-ribo fluoro R₂-Substituent [J. R. Mercer et al., J Med. Chem., 30, 670 (1987)], a 2′-arabino fluoro R₁-substituent [C. Lopez et al., Antimicrob. Agents Chemother., 17, 803 (1980); J. A. Codere et. al., J. Med. Chem., 26, 1149 (1983)] or a 2′-arabino hydroxyl R₁-substituent [M. J. Robins et al., J Med. Chem., 34, 2275 (1991); N. K. Ayusi et al., Mol Pharmacol, 31, 422 (1987); T. C. Chou et al., Antimicrob. Agents Chemother, 31, 1355 (1987)] confers resistance to glycosidic bond cleavage, which causes deactivation.

Further, in formula (1), R₃ is a substituent selected from the group consisting of hydrogen, arylcarbonyl, heteroarylcarbonyl, heterocyclocarbonyl, 1-methyl-1,4-dihydropyridyl-3-carbonyl, 3-7C cycloalkylcarbonyl, and alkylcarbonyls with a straight or branched chain having from 1 to 8 carbon atoms. Similarly, R₄ is a substituent selected from the group consisting of hydrogen, arylcarbonyl, heteroarylcarbonyl, heterocyclocarbonyl, 1-methyl-1,4-dihydropyridyl-3-carbonyl, 3-7C cycloalkylcarbonyl, and alkylcarbonyls with a straight or branched chain having from 1 to 8 carbon atoms. When R₃ and R₄ are both hydrogen, a parent compound is formed. When R₃, R₄ or both are other than hydrogen, a derivative compound is formed. It is preferred that at least one of R₃ and R₄ is hydrogen, preferably R₄ as found in the preferred embodiments of formula (1) discussed below.

Where R₃ of formula (1) is 1-methyl-1,4-dihydropyridyl-3-carbonyl and R₄ is hydrogen, a more lipophilic derivative compound is formed in which R₃ is referred to as a chemical delivery system (CDS) or a CDS moiety. CDS derivatives may have an enhanced ability to cross the blood-brain-barrier (BBB) and localize in transduced or transfected brain cells or tissue. Due to their polar nature, pyrimidine nucleosides or acyclic nucleosides such as GCV do not readily cross the BBB. Increasing the lipophilic character of the nucleosides of formula (1), while still retaining their antiviral properties, is a viable method to enhance brain localization. One of the more successful approaches currently used to design lipophilic drugs is the dihydropyridine [symbol] pyridinium salt chemical delivery system (CDS) [N. Bodor et al., Science, 214, 1370 (1981)]. This brain-targeted concept involves the coupling of a lipophilic 1-methyl-1,4-dihydropyridyl promoiety to a drug molecule through an ester or amide linkage that is readily hydrolyzed. The compounds of formula (1) wherein the R₃ substituent is a 3′-[O-(1-methyl-1,4-dihydropyridyl-3-carbonyl)] chemical delivery system (CDS) and R₄ is hydrogen cross the BBB more readily, and then undergo oxidation in a manner analogous to the NAD [symbol] NADH redox system in brain tissue. The resulting pyridinium salt is highly polar which results in its cerebral trapping leading to an elevated and sustained concentration in brain tissue. Clearance from blood is facilitated, since any oxidation product in the periphery is rapidly cleared. Hydrolysis of the ester linkage of the trapped pyridinium salt releases the parent compound of formula (1) wherein the R₃ and the R₄ substituents are hydrogen and R₁ and R₂ are as defined above, and the oxidized promoiety, trigonelline. Compounds of formula (1) wherein R₄ is hydrogen possess a free 5′-hydroxyl group which is desirable for selective phosphorylation to the 5′-monophosphate derivative by the viral encoded enzyme produced in transfected tissue or cells which results in its selected metabolic trapping thereby preventing its egress.

Thus, nucleosides containing the CDS moiety as the R₃ substituent may have increased lipophilicity and an increased ability to penetrate the population of cells. As well, nucleosides containing the CDS moiety as the R₃ substituent may have the advantage of the additional polar trapping effect described above, in addition to the selective trapping occurring as a result of the selective interaction of the expressed protein with the compound. Various other moieties may also be added to the nucleosides to increase lipophilicity and tissue penetration. For example, esterification may be used, for example, with alkoxy chains having up to 7 or 8 carbon atoms.

Of the various compounds included within formula (1) as described above, the following compounds have been found to be the most preferred when the foreign gene expresses HSV-TK: IVDU, IVDU-CDS, IVFRU, IVFRU-CDS, IVFAU, IVFAU-CDS, IVAU and IVAU-CDS. All of these preferred compounds are of formula (1) and include the substituents described below. In addition, in all of these preferred compounds, when used for diagnosis and imaging, X is an iodine, and is preferably ¹²³I, as discussed above. The other substituents of each preferred compound are as follows:

IVDU—R₁ is hydrogen, R₂ is hydrogen, R₃ is hydrogen and R₄ is hydrogen.

IVDU-CDS—R₁ is hydrogen, R₂ is hydrogen, R₃ is 1-methyl-1,4-dihydropyridyl-3-carbonyl and R₄ is hydrogen.

IVFRU—R₁ is hydrogen, R₂ is fluorine, R₃ is hydrogen and R₄ is hydrogen.

IVFRU-CDS—R₁ is hydrogen, R₂ is fluorine, R₃ is 1-methyl-1,4-dihydropyridyl-3-carbonyl and R₄ is hydrogen.

IVFAU—R₁ is fluorine, R₂ is hydrogen, R₃ is hydrogen and R₄ is hydrogen.

IVFAU-CDS—R₁ is fluorine, R₂ is hydrogen, R₃ is 1-methyl-1,4-dihydropyridyl-3-carbonyl and R₄ is hydrogen.

IVAU—R₁ is hydroxyl, R₂ is hydrogen, R₃ is hydrogen and R₄ is hydrogen.

IVAU-CDS—R₁ is hydroxyl, R₂ is hydrogen, R₃ is 1-methyl-1,4-dihydropyridyl-3-carbonyl and R₄ is hydrogen.

Of these compounds, it is preferable to use those compounds which show greater stability. For example, it has been found that of the parent compounds, IVDU is less stable than IVFRU, IVFAU and IVAU. However, it has been further found that IVDU becomes more stable as a IVDU-CDS derivative.

All of the parent and derivative compounds of formula (1), for which R₄ is hydrogen, except for those derivative compounds in which R₃ is CDS, can be prepared by reacting in an inert solvent, a 5-iodouracil nucleoside of the formula (2):

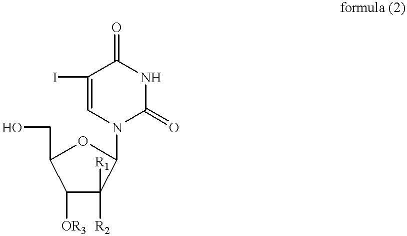

wherein R₁, R₂ and R₃ are as defined above, with (E)-1-(tri-n-butylstannyl)-2-(trimethylsilyl)ethene of formula (3):

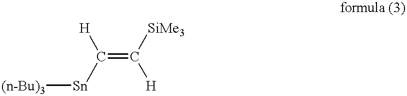

in the presence of a suitable cross-coupling catalyst, preferably of formula (4):

[(Ph)₃P]₂Pd(II)Cl₂ formula (4)

allowing the reaction to occur (normally at 50° C.) in an inert solvent such as tetrahydrofuran to convert to a (E)-5-(2-trimethylsilylvinyl)-2′-deoxyuridine or -arabinouridine compound of formula (5):

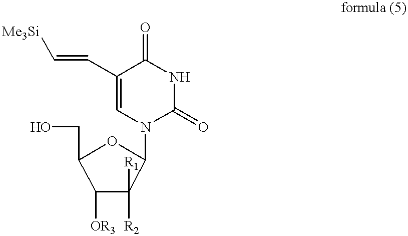

wherein R₁, R₂ and R₃ are as defined above. Electrophilic halogenation of the compound of formula (5) wherein R₁, R₂ and R₃ are as defined above using a halogenation agent of formula (6):

X—Y formula (6)

wherein X is as defined above and Y is selected from a group consisting of iodo, bromo, chloro, fluoro, acetoxy and trifluoromethoxy, in an inert solvent such as dry acetontrile, allowing the reaction to occur (normally at 25° C.) to convert to a product of formula (7):

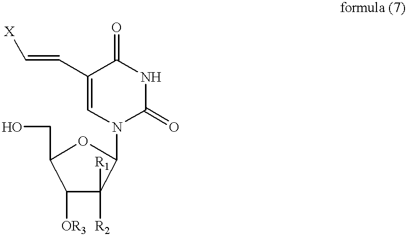

wherein X, R₁, R₂ and R₃ are as defined above.

The derivative compounds of formula (1) in which R₃ is CDS and R₄ is hydrogen can be prepared by quaternization of a compound of formula (8):

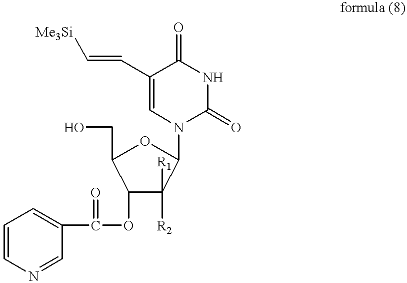

wherein R₁ and R₂ are as defined above, with iodomethane or bromomethane in an inert solvent such as acetone (preferably at reflux temperature), to convert to a compound of formula (9):

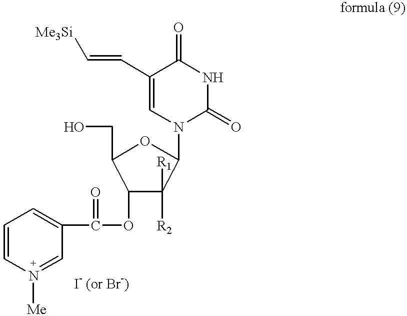

wherein R₁ and R₂ are as defined above. Electrophilic halogenation of the compound of formula (9) wherein R₁ and R₂ are as defined above using a halogenation agent of formula (10):

X—Y formula (10)

wherein X is as defined as above and Y is selected from a group consisting of iodo, bromo, chloro or fluoro, in an inert solvent such as acetonitrile, allowing the reaction to occur (normally at 25° C.) to convert to a compound of formula (11):

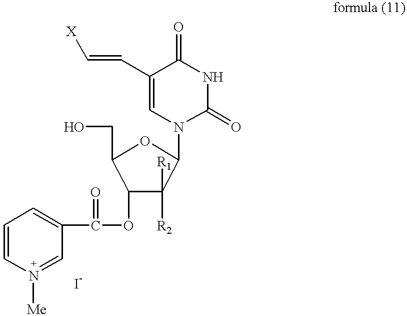

wherein X, R₁ and R₂ are as defined above. Reduction of the compound of formula (11) using a suitable reducing agent such as preferably sodium dithionite in the presence of a suitable base as sodium bicarbonate in a suitable inert two-phase solvent system (preferably water-ethyl acetate, 1:1, v/v) preferably at 25° C. to convert to a product of formula (12):

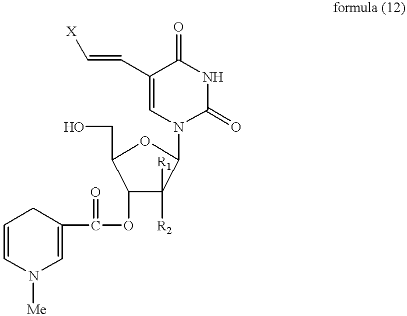

wherein X₁, R₁, and R₂ are as defined above.

The starting materials for the preparation of compounds of formula (1), viz the 5-iodouracil nucleosides of formula (2), (E)-I-(tri-n-butylstannyl)-2-trimethylsilyl)ethene of formula (3), bis(triphenylphosphine)palladium(II) chloride of formula (4) and electrophilic halogenation reagents of formula (6) are either known or are conveniently prepared from starting materials by methods known per se.

All of the compounds of formula (1) can be administered either parentally, preferably by injection, or orally. As a liquid carrier, a carrier such as water, ethyl alcohol or polyethyleneglycol, liposomes, or other physiologically acceptable solvents or dispersing liquids can be used. For oral administration, either solid or liquid carriers may be used. One commonly used solid carrier is gum acacia, but others are also suitable.

Those skilled in medical diagnostic imaging will be able to calculate an effective dose of the labelled compound for the particular use, including human use, based on their experience with other compounds carrying similar labels. However, in general, when dealing with diagnostic uses, any radiolabelled compound should be kept to a small dosage in order to avoid any toxicity to the subject.

In summary, with respect to the diagnostic method and use, the concept of gene therapy imaging is that the transferred foreign gene will code for the production of a specific, usually foreign, protein such as an enzyme, receptor or transport protein which will interact with the imaging radiopharmaceutical, being the radiolabelled compound, to produce an unique radiolabelled product. This radiolabelled product, by virtue of its physico-chemical and biochemical properties, will be distinct from the parent radiolabelled compound. The nature of these interactions may include chemical conversion to a new chemical class of compound, selective bonding via immuno-recognition, irreversible or strong binding to a transporter of enzyme protein, or other highly specific interaction. These reactions will result in differing rates of clearance of the radiolabelled compound and its radiolabelled product. For imaging, the radiolabelled compound is selected specifically to ensure that the radiolabelled product has a slower rate of clearance than the radiolabelled compound, which process is referred to as “metabolic trapping”.

The appropriate time interval or period of time between injection of the radiolabelled compound and imaging depends on, amongst other factors, the half-life of the radiolabelled compound, the rate of interaction of the radiolabelled compound with the protein to produce the radiolabelled product, the rate of clearance of the radiolabelled compound and the rate of clearance of the radiolabelled product. Thus, the time period must be particularly determined or selected for each specific gene therapy/imaging paradigm (i.e. foreign gene/labelled product/radiolabel). A time period of 1.5-24 h is most common, with the shorter periods used for ¹⁸F imaging and the longer times for radiolabels like ¹²³I. After the appropriate time period, retained radioactivity will be due to the radiolabelled product. Optimal times are selected to provide best image contrast, that is, the time when excretion of the radiolabelled compound is complete or substanially complete, and sufficient radiolabelled product remains for adequate signal measurement. A positive image will show uptake of radioactivity in a region, which reflects proof of expression of the transferred gene (i.e. measurement by imaging). Nuclear medicine imaging techniques, including planar (2-dimensional), positron emission tomography (PET) and single photon emission tomography (SPECT) imaging, and their interpretations, are known to practitioners versed in the art.

In the preferred embodiment, the transferred foreign gene is a viral thymidine kinase (HSV-1 TK) and the labelled compound is a nucleoside (IVFRU) radiolabelled with a gamma-ray (¹²³I-, ¹³¹I-) or positron-emitting (¹⁸F-; ¹²⁴I-) element. This radiolabelled compound (¹²³I-IVFRU) has been determined to be a substrate for the gene expression product (the enzyme HSV-1 TK) but not for the human TK enzyme. The radiolabelled product of the interaction between the radiolabelled compound (labelled IVFRU) and HSV-1 TK is the mono-phosphorylated nucleoside (radiolabelled IVFRU-5′-phosphate).

This radiolabelled IVFRU-monophosphate is ionic at physiological pH and cannot diffuse out of cells in which it is produced (e.g. cells that express the transferred gene), nor is it a substrate for any transmembrane transporter, whereas IVFRU itself is readily and reversibly transported into and out of cells by one or more nucleoside transporters. The overall result is the specific metabolic trapping of radioactive IVFRU-phosphate. After about a 6-8 hour period, the labelled compound, being radioactive IVFRU, will be excreted, whereas the labelled product, being radioactive IVFRU-phosphate, remains trapped in those cells expressing the transferred HSV-1 TK gene. The patient is then imaged to detect the presence of the radioactivity and delineate the region where the transferred gene, HSV-1 TK, is being expressed.

In a second embodiment of the invention, the invention is comprised of a radiotherapeutic use of a radiolabelled compound and a method of radiotherapy using that radiolabelled compound. The method and use involve a population of cells into which a foreign gene, which expresses a protein, has been transferred. The method is comprised of the step of administering to the cells an effective radiotherapeutic dose of the radiolabelled compound so that the radiolabelled compound interacts selectively with the protein to produce a radiolabelled product. The radiolabelled product achieves or performs the desired therapeutic function or effect. The radiolabelled compound is selected to interact selectively with the protein such that the radiolabelled product becomes trapped within those of the cells in which the protein has been expressed by the foreign gene. The radiotherapy method may be used in conjunction with the diagnostic and chemotherapy methods, or may be used separately.

All of the considerations, parameters, properties, methods of preparation and administration and other characteristics provided above for the population of cells, the foreign gene, the compound and the product, and the preferred embodiments of each, with respect to the diagnostic method and use are applicable to the radiotherapeutic method and use except as hereafter specified.

The population of cells of the radiotherapeutic method and use may be of the same type as the population of cells indicated for the diagnostic method and use. Specifically, at least a portion of the population of cells is preferably actively synthesizing DNA. Therefore, the most likely cells for the application of the radiotherapeutic method and use are cancerous cells or tissue or other cell proliferative conditions.

In the radiotherapeutic method and use, the compound and the product are radiolabelled as in the preferred embodiment of the diagnostic method. Thus, the X substituent of formula (1), being the radiolabel, may similarly be any suitable radioactive halogeno substituents. Suitable radioactive halogeno substituents include ¹²³I, ¹²⁵I, and ¹³¹I. Of this group, for radiotherapeutic purposes, ¹³¹I is the most preferred. It has been found that ¹²⁴I, ⁷⁵Br, ⁷⁶Br and ¹⁸F are less appropriate or suitable for radiotherapeutic purposes. Therefore in the preferred embodiment of the radiotherapeutic method and use, X in the compound of formula (1) is 131I. Further, as in the diagnostic method and use, the compound of formula (1) is preferably a high-specific-activity no-carrier-added compound.

Those skilled in medical radiotherapeutic methods and uses will be able to calculate a suitable effective dose of the radiolabelled compound for human or other uses based on their experience with other compounds carrying similar radiolabels. However, as indicated previously, when the radiolabelled compound is used for diagnostic purposes, as small a dosage as possible should be used in order to minimize any toxicity to the population of cells or surrounding tissue. When using the compound for radiotherapeutic purposes, an effective radiotherapeutic dose of the radiolabelled compound must be used. Typically, the dosage of the radiolabelled compound for therapeutic purposes will be greater than that used for diagnostic purposes in order to achieve the desired radiotherapeutic effect. When used on cancerous cells, the desired radiotherapeutic effect will be destruction of the cells in which the protein has been expressed by the foreign gene.

As discussed previously, given the manner of the foreign gene transference into the population of cells, the radiotherapeutic method and use are particularly useful when at least a portion of the population of cells is actively synthesizing DNA. Non-proliferating cells or surrounding tissue should not acquire the foreign gene and will thus remain insensitive to the radiolabelled compound. The result is that systemic toxicity should not be observed upon use of the radiolabelled compound.

In a third embodiment of the invention, the invention is comprised of a chemotherapeutic use of a compound and a method of chemotherapy using that compound. The chemotherapeutic use and method also involve a population of cells into which a foreign gene, which expresses a protein, has been transferred. The chemotherapeutic method is comprised of the step of administering to the cells an effective chemotherapeutic dose of a compound so that the compound interacts selectively with the protein to produce a product. The compound is selected to interact selectively with the protein such that the product which is produced is cytotoxic or cytostatic to those of the cells in which the protein has been expressed by the foreign gene. The chemotherapy method may be used in conjunction with the diagnostic and radiotherapy methods, or may be used separately.

All of the considerations, parameters, properties, methods of preparation and administration and other characteristics described above for the population of cells, the foreign gene, the compound and the product, and the preferred embodiment of each, with respect to the diagnostic method and use are applicable to the chemotherapeutic method and use except as hereafter specified.

As in the diagnostic method and in the radiotherapeutic method, at least a portion of the population of cells is preferably tissue actively synthesizing DNA. Therefore, again, the most likely cells for the application of the chemotherapeutic method and use are cancerous cells or tissue or other cell proliferative conditions. As indicated, the product is cytotoxic or cytostatic to the dividing cells.

In the chemotherapeutic method and use, it is not necessary that either the compound or the product be labelled. Thus, in the chemotherapeutic method and use, the X substituent of formula (1) is any suitable halogeno substituent. Suitable halogeno substituents include iodo, bromo, chloro, and fluoro. Of this group, for chemotherapeutic purposes, fluoro is the least preferred halogeno substituent.

Although the preferred embodiment of the chemotherapeutic method and use uses the herpes simplex virus gene and specifically the gene expressing HSV-TK, other genes may be employed for the chemotherapeutic method and use as long as the protein expressed by the gene is matched with an appropriate compound to produce the required product which is cytotoxic or cytostatic to the cells. For example, the human cytomegalovirus (HCMV) UL97 gene which encodes a protein that phosphorylates GCV offers potential for treating HCMV infections [E. Littler et al., Nature, 358, 160 (1992); V. Sullivan et al., Nature, 358, 162 (1992)]. In addition, transfer of genes that encode for thymidylate synthase (TS), polymerase or cytosine deaminase from eucaryotic or procaryotic organisms could be employed.

Those skilled in medical chemotherapeutic methods and uses will be able to calculate a suitable effective dose of the compound for human and other uses based on their experience with other similar compounds. As indicated previously, the transference of the foreign gene is preferably into those cells actively synthesizing DNA, so that non-proliferating cells or surrounding tissue do not acquire the foreign gene and thus remain insensitive to the chemotherapeutic compound. Thus, systemic toxicity should not be observed in use of the chemotherapeutic compound. In addition, it is known that only about 10 percent of cancerous cells need to be transduced with the HSV-TK gene to obtain effective tumor destruction using an antiviral drug such as GCV. This important observation is due to a by-stander effect where non-transduced cells are also killed. Although the mechanism of this effect is not fully understood, explanations offered to explain this phenomenon include: (i) continued viral infection; (ii) transfer of an integrated HSV-TK gene during mytosis; (iii) transfer of the toxic GCV-triphosphate via gap junctions or apoptotic vesicles; or (iv) immune related effects [S. Freeman et al., Cancer Res., 53, 5274 (1993); W. Roy Syrnthe et al., Cancer Res., 54, 2055 (1994)].

The following non-limitative examples illustrate some selective methods for producing the compounds according to the present invention, as well as comparative data illustrating the in vitro physicochemical and biological properties and in vitro biodistribution and imaging data of representative compounds according to the present invention.

### Preparation

### EXAMPLE 1

(E)-5-(2-iodovinyl)-2′-fluoro-2′-deoxyuridine (IVFRU)

(See Schematic Representation of Reaction)

Bis(triphenylphosphine)palladium(II) chloride (37 mg, 0.053 mmol) and (E)-1-(tri-n-butylstannyl)-2-(trimethylsilyl)ethene (412 mg, 1.06 mmol) were added to a solution of 5-iodo-2′-fluoro-2′-deoxyuridine (197 mg, 0.53 mmol) in dry THF (5 ml) with stirring at 50° C. The reaction mixture was stirred for 16 h at 50° C. under an argon atmosphere at which time TLC analysis indicated that the reaction was complete. The solvent was removed in vacuo and the residue obtained was purified by silica gel column chromatography using MeOH—CH₂Cl₂ (1:25, v/v) as eluent to yield (E)-5-(2-trimethylsilylvinyl)-2′-fluoro-2′-deoxyuridine as a white solid (146 mg, 80% yield) after recrystallization from ethyl acetate; mp 164-165° C., ¹H NMR (DMSO-d₆):δ 0.08 (s, 9H, SiMe₃), 3.64 (m, 1H, J_{gem}=9.9 Hz, H-5′), 3.86 (m, 1H, H-5″), 3.89 (d, 1H, J_{3′4′}=8.8 Hz, H-4′), 4.21 (m, 1H, J_{3′,f}=24.2 Hz, H-3′), 5.04 (dd, 1H, J_{2′.F}=53.3 Hz, J_{2′,3′}=3.8 Hz, H-2′), 5.43 (t, 1H, J_{OH,3′}=6.6 Hz, C-3′ OH, exchanges with deuterium oxide), 5.92 (d, 1H J_{1′,F}=17.0 Hz, H—I′), 6.52 and 6.59 (two d, 1H each, Jₜᵣₐₙₛ=19.8 Hz, CH═CHSiMe₃), 8.33 (s, 1H, uracil H-6), 11.51 (s, 1H, NH, exchanges with deuterium oxide); Exact Mass Calcd for C₁₄H₂₁FN₂O₅Si:344.1204. Found (HRMS): 344.1208 (M⁺, 2.2%); Anal. Calcd for C₁₄H₂₁FN₂O₅Si:C, 48.82; H, 6.15; N, 813. Found: C, 48.68; H, 6.16; N, 7.87.

(E)-5-(2-trimethylsilylvinyl)-2′-fluoro-2′-deoxyuridine (40 mg, 0. 116 mmol) was dissolved in acetonitrile (2 MI) and then iodine monochloride (19 mg, 0. 116 mmol) was added with stirring at 25° C. The reaction mixture was stirred for 30 min at 25° C. at which time TLC analysis indicated that the reaction was completed. The solvent was removed in vacuo and the residue obtained was purified by silica gel column chromatography using MeOH—CH₂CI₂ (7:93, v/v) as eluent to afford (E)-5-(2-iodovinyl)-2′-fluor-2′-deoxyuridine (IVFRU, 36 mg, 78% yield) as a white solid after recrystallization from methanol; mp 108-110OC; ¹H NMR (DMSO-d₆):δ 3.65 (d, 1H, J_{gem}=12 Hz, H-5′), 3.82-3.96 (m, 2H, H-4′, H-5″), 4.20 (dd, 1H, J_{3′,F}=23 Hz, J_{2..3′}=6 Hz, H3′), 5.06 (dd, 1H, J_{2′,F}=54 Hz, J_{2′,3′}=6 Hz, H-2′), 5.47 (br s, 1H, C-5′ OH, exchanges with deuterium oxide), 5.72 (br s, 1H, C-3′ OH, exchanges with deuterium oxide), 5.92 (d, 1H, J_{1,F}18 Hz, H-1′), 7.09 (d, 1H, Jₜᵣₐₙₛ=16 Hz, CH═CHI), 7.20 (d, 1H, Jₜᵣₐₙₛ=16 Hz, CH═CHI), 8.23 (s, 1 H, uracil H-6), 11.6 (br s, 1 H, NH, exchanges with deuterium oxide); Exact Mass Calcd for C₁₁H₁₂FIN₂O₅: 397.9775. Found (HRMS): 397.9773 (M⁺, 3.6%); Anal. Calcd. for C₁₁H₁₂FIN₂O₅: C, 33.19; H, 3.04; N, 7.04. Found: C, 33.83; H, 3.32; N, 7.00.

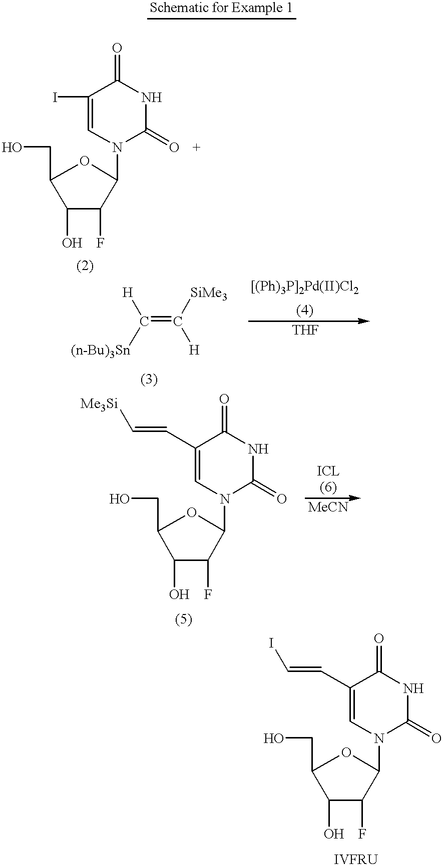

### EXAMPLE 2

The related (E)-5-(2-iodovinyl)-2′-fluoro-2-deoxyarabinouridine (IVFAU), (E)-5-(2-iodovinyl)arabinouridine (IVAU), and (E)-5-(2-iodovinyl)-2′-deoxyuridine (1VDU) compounds have been prepared, using a procedure similar to that used in Example 1, as illustrated in the schematic for Example 2 shown below using an equivalent quantity of the (E)-5-iodouracil nucleoside of formula (2), in place of (E)-5-iodo-2′-fluoro-2′-deoxyuridine in Example 1, to afford IVFAU, IVAU and IVDU which had melting points of 176-178° C., 171-175° C. and 166-170° C., respectively.

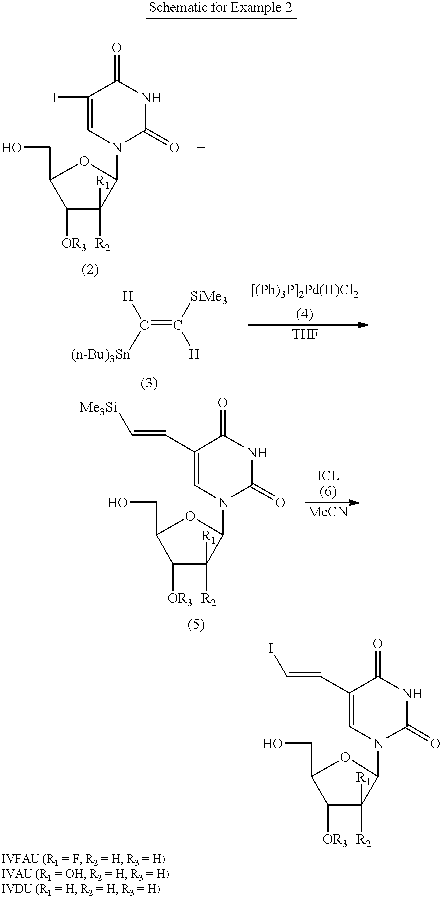

### EXAMPLE 3

Carrier Added Synthesis of [¹³¹I]-(E)-5-(2-iodovinyl)-2′-fluoro-2′-deoxyuridine {[¹³¹I-IVFRU}

(See Schematic Presentation Following Example)

A solution of [¹³¹I]-Nal (74 MBq) in 0.1 N NaOH (5 μL) was placed in a Wheaton vial and then a solution of ICI (124 μg, 0.765 μmol) in acetic acid-acetonitrile (1:4, v/v; 10 μL) was added. A solution of (E)-5-(2-trimethylsilylvinyl)-2′-fluoro-2′-deoxyuridine (500 μg, 1.53 μmol) in acetic acid-acetonitrile (1:4, v/v, 10 μL) was then added to the contents in the Wheaton vial and the reaction was allowed to proceed for 15 min at 25° C. The product was purified by preparative reverse phase HPLC using a Whatman Partisil M9 10/25 C8 column by isocratic elution with acetonitrile-water (70:30, v/v) at a flow rate of 1.5 MI/min. The product [¹³¹I]-IVFRU had a retention time of 11.76 min under these conditions whereas unreacted (E)-5-(2-trimethylsilylvinyl)-2′-fluoro-2′-deoxyuridine had a retention time of 24.19 min. [¹³¹I]-(E)-5-(2-iodovinyl)-2′-fluoro-2′-deoxyuridine (63 Mbq, 85% radiochemical yield, >98% radiochemical purity, specific activity 252 GBq/mmol) prepared using this procedure displayed identical chromatographic retention times to that observed with an authentic sample of unlabelled IVFRU under a variety of chromatographic conditions.

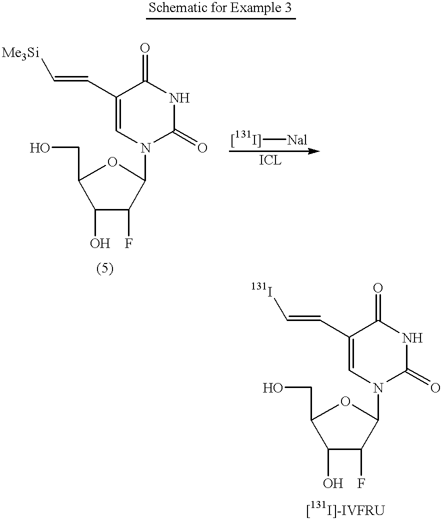

### EXAMPLE 4

No Carrier Added Synthesis of [¹³¹I]-(E)-5-(2-iodovinyl)-2′-fluoro-2′-deoxyuridine {[¹³¹I]-IVFRU}

(See Schematic Presentation Following Example)

A solution of (E)-5-(2-trimethylsilylvinyl)-2′-fluoro-2′-deoxyuridine (100 μg, 0.306 μmol) in acetic acid-acetonitrile (1:4, v/v, 10 μL) was added to a solution of [¹³¹I]-Nal (11.3 Mbq) in 0. 1 N NaOH (5 μL) in a Wheaton vial. A solution of N-chlorosuccinimide (100 μg, 0.749 μmol) in acetic acid-acetonitrile (1:4, v/v, 10 μL) was then added, the reaction was allowed to proceed for 30 min at 25° C., and the reaction was terminated by the addition of sodium thiosulfate (100 μg, 0.632 μmol) in water (10 μL). The reaction mixture was separated by HPLC using the procedure described in Example 3 to afford [¹³¹I]-(E)-5-(2-iodovinyl)-2′-fluoro-2′-deoxyuridine (8.0 Mbq, 71% radiochemical yield, >98% radiochemical purity, specific activity >5.29 TBq/mmol) as a no carrier added product.

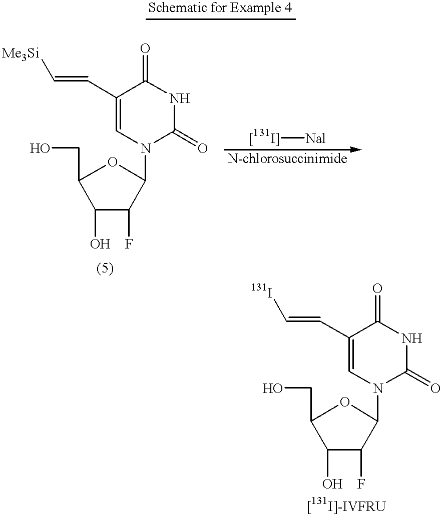

### EXAMPLE 5

The related [¹²³I]-, [¹²⁴I]-, [¹²⁵I]- and [¹³¹]-labelled IVFAU, IVAU and IVDU compounds can be prepared, using a procedure similar to that used in Example 3 using an equivalent quantity of the (E)-5-(2-trimethylsilylvinyl)uracil nucleoside of formula (5), in place of (E)-5-(2-trimethylsilylvinyl)-2′-fluoro-2′-deoxyuridine in Example 3, to afford [¹²³I]-, [¹²⁴I]-, [¹²⁵I]- and [¹³¹]-labelled IVFAU, IVAU and IVDU. For example, [¹²⁵I]-VDU was prepared using this procedure (59% radiochemical yield, >98% radiochemical purity, specific activity of 12.7 Gbq/mmol).

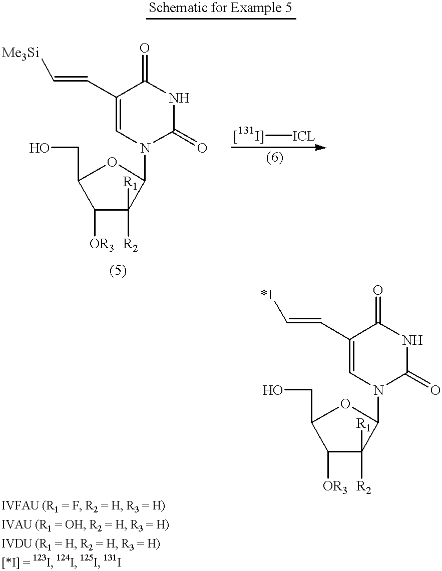

### EXAMPLE 6

(E)-5-(2-iodovinyl)-3′-O-(1-methyl-1,4-dihydropyridyl-3-carbonyl)-2′-fluoro-2′-deoxyuridine (IVFRU-CDS)

(See Schematic Presentation Following Example)

Iodomethane (165 mg, 1.16 mmol) was added to a solution of (E)-5-(2-trimethylsilylvinyl)-3′-O-(3-pyridylcarbonyl)-2′-fluoro-2′-deoxyuridine (26 mg, 0.058 mmol) in Acetone (3 MI) and the reaction mixture was heated at reflux for 16 h. Removal of the solvent in vacuo and trituration of the residue obtained with ether (3×10 MI) gave (E)-5-(2-trimethylsilylvinyl)-3′-O-(1-methylpyridinium-3-carbonyl)-2′-fluoro-2′-deoxyuridine iodide (31 mg, 90%) as yellow crystals, mp 172-174° C.; 1H NMR (DIVISO-d₆: δ 0.1 (s, 9H, SiM₃), 3.7-3.9 (m, 2H, H-5′), 4.44 (br s, 4H, NCH₃, H-4′), 5.44 (t, 1H, J_{OH.5′}=3 Hz, C-5′ OH, exchanges with deuterium oxide), 5.55-5.58 (m, 1H, H-3′), 5.58-5.80 (m, 1H, H-2′), 6.12 (dd, 1H, J_{1′,F}=17.0, J_{1′,2′}=1.8 Hz, H-1′), 6.60 (s, 2H, CH═CHTMS), 8.22 (s, 1H, uracil H-6), 8.29 (dd, 1H, J_{4,5}=7.6, J_{5,6}=5.0 Hz, pyridinium H-5), 9.06 (d, 1H, J_{4,5}=7.6 Hz, pyridinium H-4), 9.23 (d, 1H, J_{5,6}=5.0 Hz, pyridinium H-6), 9.66 (s, 1H, pyridinium H-2), 11.62 (s, 1H, NH, exchanges with deuterium oxide). Anal. Calcd for C₂₁H₂₇FIN₃O₆Si.1/2 H₂O: C, 42.00; H, 4.71; N, 6.99. Found: C, 42.37; H, 4.57; N, 6.57.

Iodine monochloride (1.7 mg, 0.01 mmol) was added to a solution of (E)-5-(2-trimethylsilylvinyl)-3′-O-(1-methylpyridinium-3-carbonyl)-2′-fluoro-2′-deoxyuridine iodide (6 mg, 0.0101 mmol) in acetonitrile (1 MI), immediately after its preparation in the previous reaction, and the reaction mixture was stirred at 25° C. for 15 min. Removal of the solvent in vacuo gave (E)-5-(2-iodovinyl)-3′-O-(1-methylpyridinium-3-carbonyl)-2′-fluoro-2′-deoxyuridine iodide as a yellow solid which was used immediately without further purification in the subsequent reaction. This yellow solid was dissolved in a two phase solvent system comprised of water-ethyl acetate (1 MI each), sodium dithionite.(10 mg, 0.057 mmol) and sodium bicarbonate (4 mg, 0.048 mmol) were added and the reaction was allowed to proceed at 25° C. with stirring for 15 min. The ethyl acetate fraction was washed with water (1 MI) and the ethyl acetate solution was dried (Na₂SO₄). The solvent was removed in vacuo and the product was purified using a short neutral aluminum oxide column with chloroform-methanol (90:10, v/v) as eluent to afford (E)-5-(2-iodovinyl)-3′-O-(1-methyl-1,4-dihydropyridyl-3-carbonyl)-2′-fluoro-2′-deoxyuridine (IVFRU-CDS, 3 mg, 59%) as a yellow solid after recrystallization from methanol, mp 131-133° C.; ¹H NMR (CDCI₃): μ3.02 (s, 3H, NCH₃), 3.11 (br s, 2H, dihydropyridyl H-4), 3.86 and 4. 10 (two d, 1H each, J_{gem}=12.5 Hz, H-5′), 4.27 (d, 1H, J_{3′,4′}=7.5 Hz, H-4′), 4.87 (dt, 1H, J_{5.6}=8.0, J_{4,5}=3.8 Hz, dihydropyridyl H-5), 5.18 (m, 1H, J_{2′,F}=51 Hz, H-2′), 5.32 (m, 1H, H-3′), 5.68 (d, 1H, J_{5.6}=8.0 Hz, dihydropyridyl H-6) 6.05 (d, 1H, J_{1′,F}=16 Hz, H-1′), 7.06 (d, 1H, Jₜᵣₐₙₛ=15 Hz, CH═CHI), 7.11 (s, 1H, dihydropyridyl H-2) 7.40 (d, 1H, Jₜᵣₐₙₛ=15 Hz, CH═CHI), 8.12 (s, 1H, uracil H-6). Anal. Calcd. for C₁₈H₁₉FIN₃O₆. H₂O: C, 40.24; H, 3.94; N, 7.82. Found: C, 40.52; H, 4.09; N, 7.61.

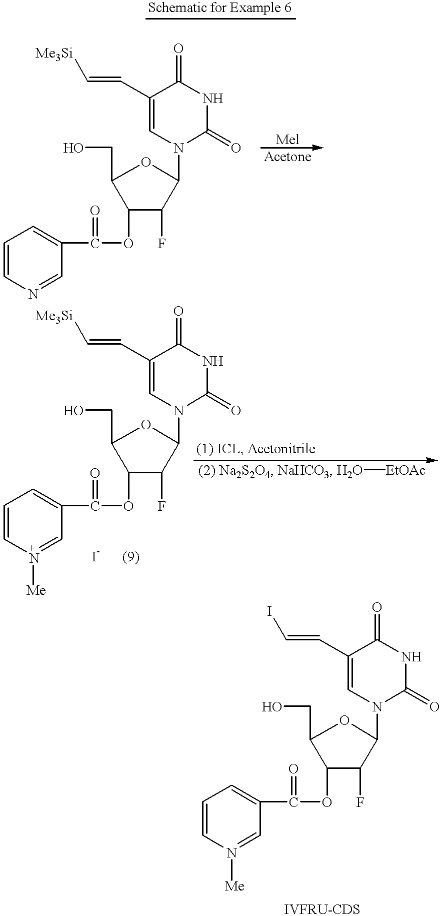

### EXAMPLE 7

The related (E)-5-(2-iodovinyl)-3′-O-(1-methyl-1,4-dihydropyridyl-3- carbonyl)-2′-fluoro-2′-deoxyarabinouridine (IVFAU-CDS) and (E)-5-(2-iodovinyl)-3′-O-(1-methyl-1,4-dihydropyridyl-3-carbonyl)-2′-deoxyuridine (IVDU-CDS) compounds have been prepared by a procedure similar to that used in Example 6 using an equivalent quantity of a (E)-5-(2-trimethylsilylvinyl)uracil nucleoside of formula (9), in place of (E)-5-(2-trimethylsilylvinyl)-3′-O-(3-pyridylcarbonyl)-2′- fluoro-2′-deoxyuridine in Example 6, to afford IVFAU-CDS and IVDU-CDS with melting points of 148-152° C. and 165-168° C., respectively.

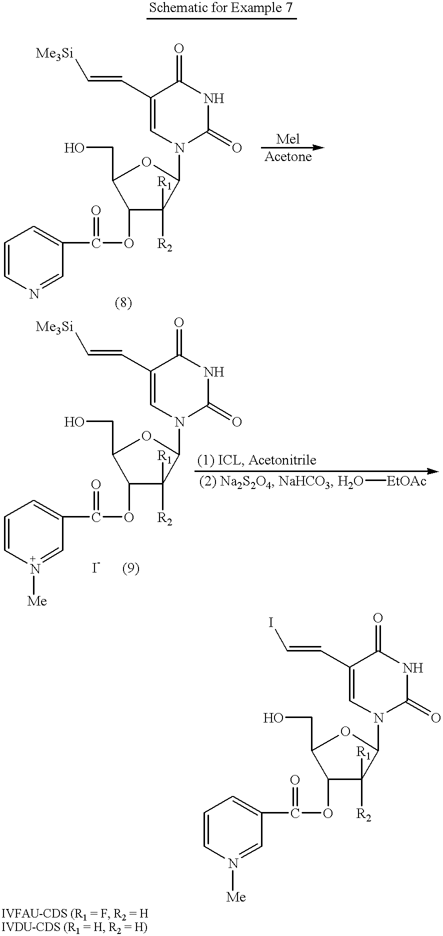

### EXAMPLE 8

Carrier Added Synthesis of [¹³¹I]-(E)-5-(2-iodovinyl)-3′-O-(1-methyl-1,4-dihydropyridyl-3-carbonyl)-2′-fluoro-2′-deoxyuridine {[¹³¹I]-IVFRU-CDS}

(See Schematic Presentation Following Example)

A solution of (E)-5-(2-trimethylsilylvinyl)-3′-O-(1-methylpyridinium-3-carbonyl)-2′-fluoro-2′-deoxyuridine bromide (1 mg, 0.00184 mmol) was dissolved in acetonitrile (100 μL) which was immediately added to a stirred solution of Icl (30 μg, 0.185 mol) and [¹³¹I]-Nal (26.8 Mbq) in acetonitrile (20 μL). The reaction was allowed to proceed for 15 min at 25° C., the solvent was evaporated over a stream of nitrogen gas and the residue obtained was then dissolved in degassed water (200 μL) and ethyl acetate (200 μL). Sodium dithionite (4 mg, 0.0229 mmol) and sodium bicarbonate (2 mg, 0.0238 mmol) were added and the reaction was allowed to proceed with stirring for 20 min at 25° C. The ethyl acetate layer was then separated and the solvent was evaporated over a stream of nitrogen. The residue obtained was dissolved in methanol and purified by preparative reverse phase HPLC using a Whatman Partisil M9 10/25 C8 column. Isocratic elution with acetonitrile:water (60:40, v/v) at a flow rate of 2.0 MI/min gave pure [¹³¹]-(E)-5-(2-iodovinyl)-3′-O-(1-methyl-1,4-dihydropyridyl-3-carbonyl)-2′-fluroro-2′-deoxyuridine {[¹³¹I]-IVFRU-CDS}, retention time of 19.54 min, in 14% isolated radiochemical yield (3.8 Mbq), having a specific activity of 4.3 Gbq/mmol and >98% radiochemical purity after HPLC purification.

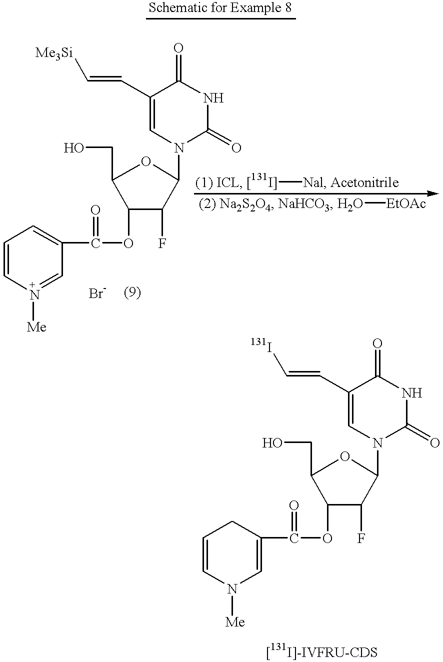

### EXAMPLE 9

The related [¹²³I], [¹²⁵I]-, [125I]- and [¹³¹I]-labelled IVFAU-CDS, IVAU-CDS and IVDU-CDS compounds can be prepared, using a procedure similar to that used in Example 8 using an equivalent quantity of the (E)-5-(2-trimethylsilylvinyl)uracil nucleoside of formula (10), in place of (E)-5-(2-trimethylsilylvinyl)-3′-O-(1-methylpyridinium-3-carbonyl)-2′-fluoro-2′-deoxyuridine bromide in Example 8, to afford [¹²³I]-, [¹²⁴I]-, [¹²⁵I]- and [¹³¹I]-labelled IVFAU-CDS, IVAU-CDS and IVDU-CDS. For example, [¹³¹I]-IVDU-CDS was prepared using this procedure (59% radiochemical yield, >98% radiochemical purity, specific activity of 12.7 Gbq/mmol).

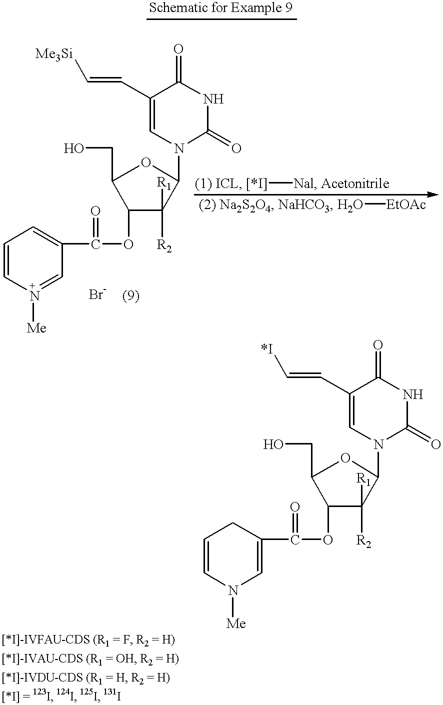

### Biological Evaluations Pertinent to the Invention

### EXAMPLE 10

Partition Coefficients and Pseudo-first Order Oxidation Rate Constants

Partition coefficients (log P) were measured by determining the distribution of the test compound between a presaturated mixture of n-octanol and water (1:1, v/v). The analytical method consisted of a modified shake-flask technique and ultraviolet (UV) spectrometry quantitation after centrifugal separation of the two phases. The concentration of the test compound in the octanol phase prior to distribution was 0.5 mM. The dihydropyridine chromophore (λmax=360 nm) was used for quantitative UV analysis of IVDU-CDS and IVFRU-CDS, for which the results are shown in Table 1. The log P values for IVDU-CDS and IVFRU-CDS were significantly higher than those for IVDU (log P=1.10) and IVFRU (log P=1.21).

Pseudo-first order rate constants and half-lives for the oxidation of IVDU-CDS and IVFRU-CDS were determined in 50% mouse blood, 20% mouse brain homogenate and 20% mouse liver homogenate. A solution of the test compound in DMSO (10 Mm) was added to each matrix and incubated at 37° C. At various times after test compound addition, an aliquot was removed and added to acetonitrile (100 μL). The samples were immediately centrifuged and the supernatant was analyzed by quantitative reverse phase HPLC. The rate of disappearance of the 1-methyl-1,4-dihydropyridine-CDS was determined by UV detection at 360 nm. The results, which are illustrated in Table 1, demonstrate that the 1-methyl-1,4-dihydropyridine promoiety undergoes facile oxidation to a pyridinium salt in selected biological tissues and fluids.

TABLE 1
Oxidation Rates in Mouse Tissues and Partition Coefficients

| | k × 10⁻³ min⁻¹ | | | t_{1/2} (min) | | | |
|---|---|---|---|---|---|---|---|
| Compound | blood | brain | liver | blood | brain | liver | log P |
| IVDU-CDS | 5.22 | 9.61 | 10.81 | 133 | 72 | 3 | 1.77 |
| IVFRU-CDS | 2.70 | 3.10 | 5.31 | 256 | 223 | 130 | 1.83 |

### EXAMPLE 11

### In Vitro Antiviral Activity Against Herpes Viruses

The in vitro antiviral activities for some selected test compounds against herpes simplex virus type 1 (HSV-1) and herpes simplex virus type-2 (HSV-2) have been determined using a cytopathic effect (CPE) inhibition assay using cultured human foreskin fibroblasts (HFF). Antiviral activity against varicella zoster virus (VZV) has been determined using a plaque reduction assay. A cell proliferation assay using uninfected HFF cells was employed for the cytotoxicity assay. The results indicated that the test compounds are potent antiviral compounds against a battery of herpes viruses, and that the test compounds exhibited minimal host cell toxicity.

### KBALB Cell Models for Gene Therapy Studies

The KBALB sarcoma models used in these studies are derived by exposing wild-type KBALB cells to Moloney murine leukemia virus (MMLV) producer cell supernatants containing replication incompetent ecotropic retroviruses. The KBALB-STK cell line was transduced with a vector possessing the HSV-1 TK and neomycin resistance genes, whereas the KBALB-LNL cells were transduced with a vector possessing only the neomycin resistance gene. These transduced cell lines are cultured in media containing the antibiotic G-418 to select for those cells expressing the neomycin resistance gene. These rapidly growing sarcomas have been characterized extensively and do not produce replication competent retrovirus particles [S. Freeman et al., Cancer Res., 53, 5274 (1993)].

### EXAMPLE 12

### In Vitro Cellular Uptake of Radiopharmaceuticals in Transduced or Non-transduced Sarcoma Cell Lines

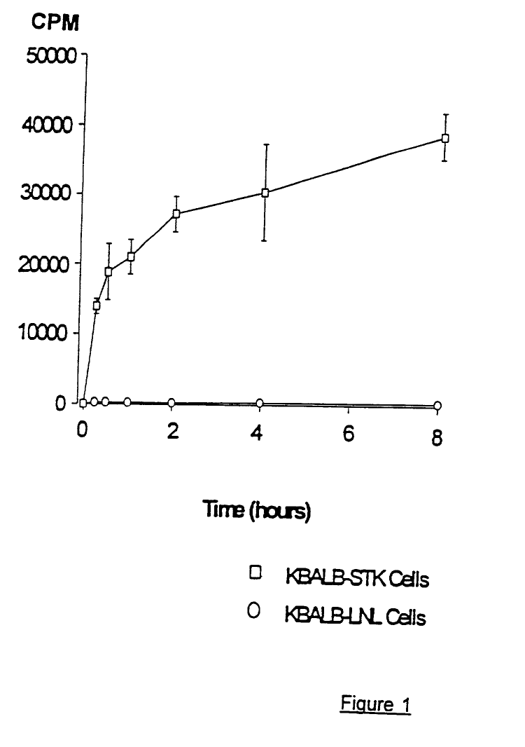

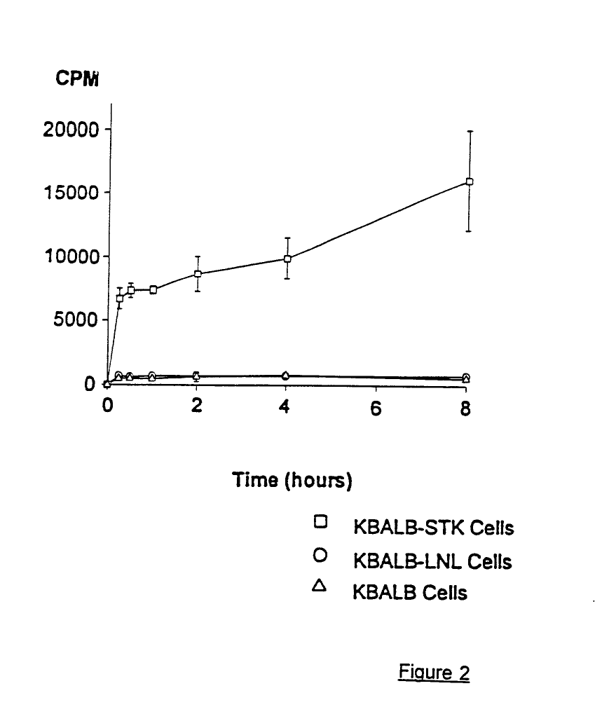

KBALB-STK, KBALB-LNL, or wild type KBALB cells were grown to confluency in 24 well culture plates. [¹²⁵I]-IVDU (sp. act.=12 Gbq/mmol), [¹²⁵I]-1VFRU (sp. act.=11 Gbq/mmol), [¹³¹′I]-IVDU-CDS (sp. act.=2.8 Gbq/mmol) or [¹³¹I]-IVFRU-CDS (sp. act.=4.3 Gbq/mmol) were added to each well in 100 μL saline solution and incubated at 37° C. At varying times after exposure to the test compound, the supernatant was removed, the cells rinsed with saline, and the adherent cells were then trypsinized prior to their removal. Cellular uptake -was determined by gamma counting using a Beckmann 8000 gamma counter. The results, which are presented in FIGS. 1 and 2, indicate that selective uptake occurred for [¹²⁵I]-VDU and [¹²⁵I]-IVFRU in KBALB-STK cells that express HSV-1 TK relative to KBALB or KBALB-LNL cells.

### EXAMPLE 13

### Biodistribution of [¹³¹I]-IVFRU in Mice Bearing KBALB or KBALB-STK Tumors

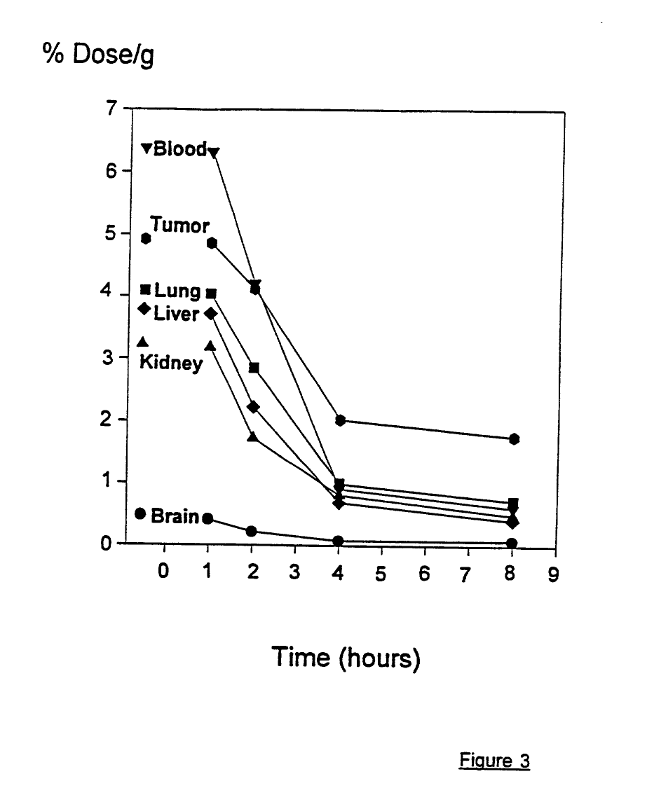

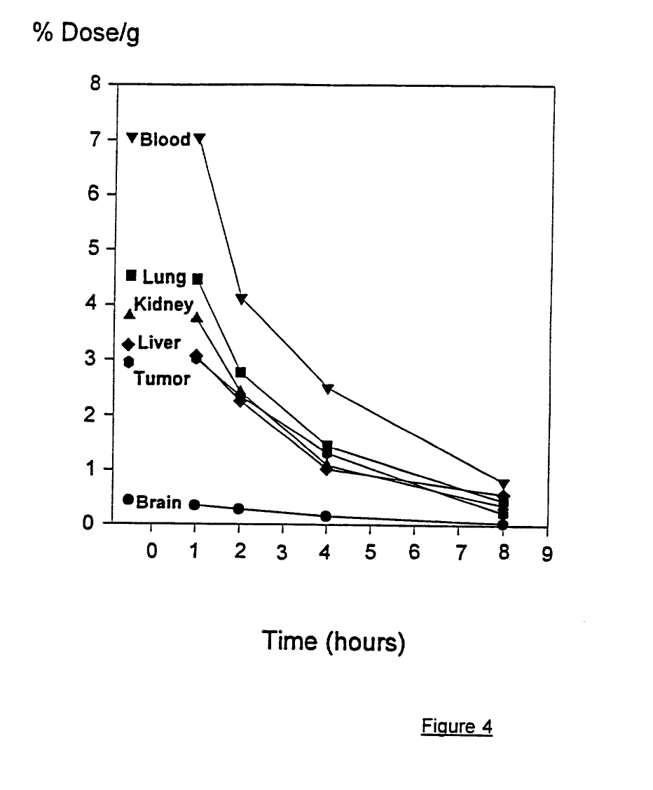

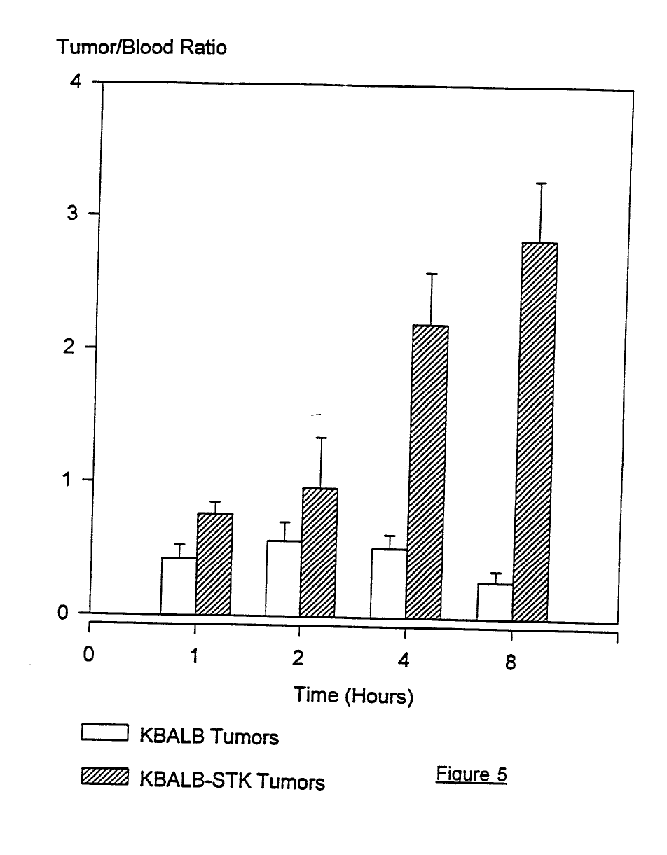

KBALB or KBALB-STK cells (1×10⁵ cells) were injected subcutaneously into the flank of male Balb-c mice. Palpable tumors appeared 14 days after injection. [¹³¹I]-IVFRU (370 KBq, sp. act. 59 Gbq/mmol) was injected via the tail vein into each tumor bearing animal. Three animals were sacrificed and dissected at each time interval. The radioactivity uptake by selected tissues of interest were determined using a Beckman 8000 gamma counter. Tissue uptake is expressed as a percentage of the administered dose per gram of tissue versus time in FIGS. 3 and 4. The tumor to blood ratio is presented for both tumor models in FIG. 5. Preferential tumor uptake is evident in tumors expressing HSV-1 TK resulting in a peak tumor/blood ratio of approximately 3 for animals bearing KBALB-STK tumors at 8 hours post injection. In contrast, wild type KBALB tumor bearing animals showed significantly less tumor radioactivity and much lower tumor/blood ratios were observed at all time points relative to those for KBALB-STK tumors.

### EXAMPLE 14

### Scintigraphic Imaging of KBALB-STK Tumors

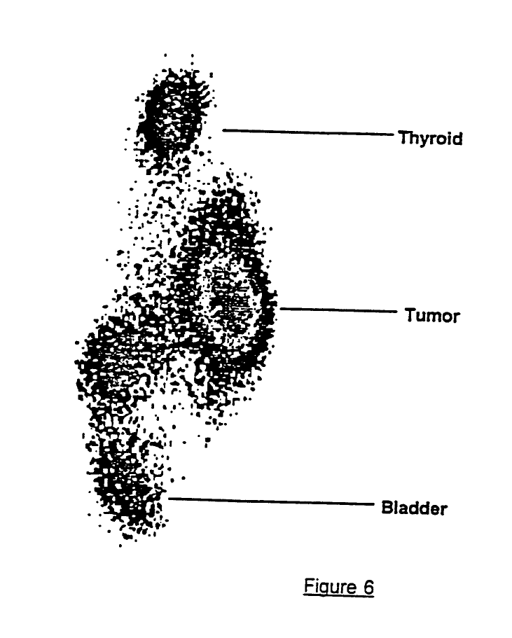

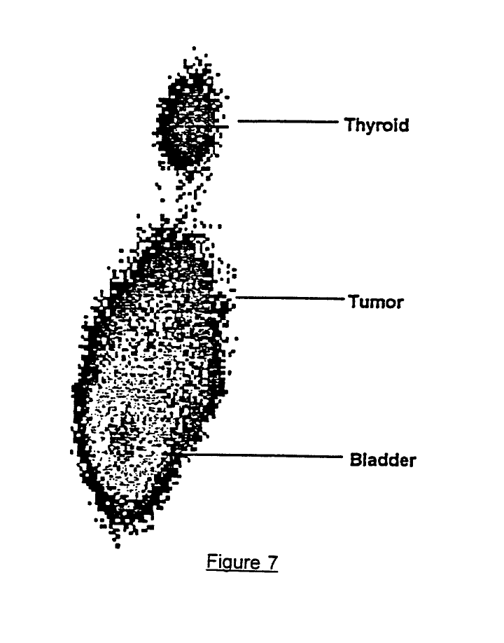

BALB-STK cells (1×10⁵ cells) were injected subcutaneously into the flank of 12 Balb-c mice. All inoculated animals developed palpable tumors suitable for imaging after 14 days at which time the tumors were approximately 10 mm in diameter. Static images were obtained following injection of 3.7 Mbq of [¹³¹I]-VFRU (sp. act.=252 Gbq/mmol) via the tail vein. Animals were placed in the prone position under a pinhole collimator using a Searle gamma camera (Scintiview) with data manipulated on an ADAC computer (DPS 3300). Images were acquired over 15 minutes using a 256×256 matrix. After initial imaging, a group of animals (n=6) were administered an intraparietoneal (ip) injection of ganciclovir (100 mg/kg) once daily for seven consecutive days. The control group (n=6) received daily saline injections. After four days, the animals were administered [¹³¹I]-IVFRU as described previously and imaging was performed using the same acquisition protocol described above. It was noted that the treatment group tumor size had shrunk to an average size of less than 5 mm. After 7 days of ganciclovir treatment, the KBALB-STK tumors had completely regressed in all treated animals. The tumors in the saline administered control animals continued to grow until termination of the experiment. The scintigram, presented as FIG. 6, for an animal prior to ganciclovir treatment, which was imaged 8 hours after [131I]-IVFRU administration, illustrates selective uptake of [131I]-IVFRU into KBALB-STK tumors expressing HSV-1 TK. The scintigram, presented as FIG. 7, is for the same animal 8 hours after [¹³¹I]-IVFRU administration following 4 days of ganciclovir treatment. The majority of the radioactivity present in the scintigram image shown in FIG. 7 was present in the urinary bladder, since the mouse had not voided, while the region of the shrinking tumor remnant was relatively devoid of activity.

The embodiments of the invention in which an exclusive property or privilege is claimed are defined as follows:

1. A method for monitoring the transfer of a foreign gene throughout a population of cells, comprising the following steps:
(a) selecting a foreign gene which has been isolated from a cell or virus and which has been transferred into the population of cells;
(b) selecting a labelled compound which will interact selectively with a protein expressed by the foreign gene to produce a labelled product and which has a rate of expulsion from the cells which is greater than a rate of expulsion from the cells of the labelled product;
(c) administering to the cells an effective dose of the labelled compound such that the labelled compound selectively interacts with the protein to produce the labelled product;
(d) waiting a period of time such that a substantial amount of the labelled compound has been expelled from the cells and such that a detectable amount of the labelled product remains within the cells; and
(e) determining the extent and location of the protein throughout the population of cells by detecting the labelled product.

2. The method as claimed in claim 1, further comprising the steps of isolating the selected foreign gene from a cell or virus and transferring the isolated foreign gene into the population of cells.

3. The method as claimed in claim 1, wherein the protein expressed by the foreign gene is not naturally occurring within the cells.

4. The method as claimed in claim 1, wherein step (d) is comprised of non-invasively detecting the labelled product.

5. The method as claimed in claim 4, wherein the labelled compound is a radiolabelled compound which interacts with the protein expressed by the foreign gene to produce a radiolabelled product which can be detected using nuclear medicine imaging techniques.

6. The method as claimed in claim 5, wherein the foreign gene is a gene selected from eucaryotic or procaryotic cells.

7. The method as claimed in claim 5, wherein the foreign gene is selected from a virus.

8. The method as claimed in claim 7, wherein the foreign gene is selected from the group of viruses consisting of herpes simplex virus, human cytomegalovirus, varicella zoster virus and Epstein-Barr virus.

9. The method as claimed in claim 8, wherein the foreign gene is a gene which expresses herpes simplex virus thymidine kinase.

10. The method as claimed in claim 9, wherein the radiolabelled compound is a compound of the formula: 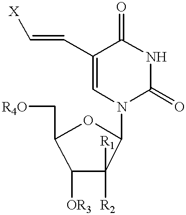
or a pharmaceutically acceptable salt thereof, wherein X is a radioactive halogeno substituent, wherein R₁ is a hydrogen, hydroxyl or fluoro substituent, wherein R₂ is a hydrogen or fluoro substituent, wherein R₃ is a substituent selected from the group consisting of hydrogen, arylcarbonyl, heteroarylcarbonyl, heterocyclocarbonyl, 1-methyl-1,4-dihydropyridyl-3-carbonyl, 3-7C cycloalkylcarbonyl, and alkylcarbonyls with a straight or branched chain having from 1 to 8 carbon atoms, and wherein R₄ is a substituent selected from the group consisting of hydrogen, arylcarbonyl, heteroarylcarbonyl, heterocyclocarbonyl, 1-methyl-1,4-dihydropyridyl-3-carbonyl, 3-7C cycloalkylcarbonyl, and alkylcarbonyls with a straight or branched chain having from 1 to 8 carbon atoms.

11. The method as claimed in claim 10, wherein X is a radioactive halogeno substituent selected from the group consisting of ¹²³I, ¹²⁴I, ¹³¹I, ⁷⁵Br, ⁷⁶Br and ¹⁸F.

12. The method as claimed in claim 11, wherein X is ¹²³I.

13. The method as claimed in claim 10, wherein X is a radioactive halogeno substituent selected from the group consisting of ¹²³I, ¹²⁴I and ¹³¹I.

14. The method as claimed in claim 13, wherein R₄ is hydrogen, wherein R₂ is hydrogen, wherein R₃ is hydrogen, and wherein R₄ is hydrogen.

15. The method as claimed in claim 14, wherein X is ¹²³I.

16. The method as claimed in claim 13, wherein R₁ is hydrogen, wherein R₂ is hydrogen, wherein R₃ is 1-methyl-1,4-dihydropyridyl-3-carbonyl, and wherein R₄ is hydrogen.

17. The method as claimed in claim 16, wherein X is ¹²³I.

18. The method as claimed in claim 13, wherein R₁ is hydrogen, wherein R₂ is fluorine, wherein R₃ is hydrogen, and wherein R₄ is hydrogen.

19. The method as claimed in claim 18, wherein X is ¹²³I.

20. The method as claimed in claim 13, wherein R₁ is hydrogen, wherein R₂ is fluorine, wherein R₃ is 1-methyl-1,4-dihydropyridyl-3-carbonyl, and wherein R is hydrogen.

21. The method as claimed in claim 20, wherein X is ¹²³I.

22. The method as claimed in claim 13, wherein R₁ is fluorine, wherein R₂ is hydrogen, wherein R₃ is hydrogen, and wherein R₄ is hydrogen.

23. The method as claimed in claim 22, wherein X is ¹²³I.

24. The method as claimed in claim 13, wherein R₁ is fluorine, wherein R₂ is hydrogen, wherein R₃ is 1-methyl-1,4-dihydropyridyl-3-carbonyl, and wherein R₄ is hydrogen.

25. The method as claimed in claim 24, wherein X is ¹²³I.

26. The method as claimed in claim 13, wherein R₁ is hydroxyl, wherein R₂ is hydrogen, wherein R₃ is hydrogen, and wherein R₄ is hydrogen.

27. The method as claimed in claim 26, wherein X is ¹²³I.

28. The method as claimed in claim 13, wherein R₁ is hydroxyl, wherein R₂ is hydrogen, wherein R₃ is 1-methyl-1,4-dihydropyridyl-3-carbonyl, and wherein R₄ is hydrogen.

29. The method as claimed in claim 28, wherein X is ¹²³I.

30. The method as claimed in claim 10, wherein at least one of R₃ and R₄ is hydrogen.

31. The method as clamed in claim 10, wherein R₄ is hydrogen.

32. A use of a labelled compound to monitor the transfer of a foreign gene throughout a population of cells, by selecting a foreign gene which has been isolated from a cell or virus and which has been transferred into the population of cells, selecting a labelled compound which will interact selectively with a protein expressed by the foreign gene to produce a labelled product and which has a rate of expulsion from the cells which is greater than a rate of expulsion from the cells of the labelled product, administering to the cells an effective dose of the labelled compound such that the labelled compound selectively interacts with the protein to produce the labelled product, waiting a period of time such that a substantial amount of the labelled compound has been expelled from the cells and such that a detectable amount of the labelled product remains within the cells, and determining the extent and location of the protein throughout the population of cells by detecting the labelled product.
